(19) United States
(12) Patent Application Publication
(10) Pub. No.: US 2025/0325367 A1
(43) Pub. Date: Oct. 23, 2025

(54) SHEATH AND LOADER ASSEMBLY FOR DELIVERING A PROSTHETIC DEVICE

(71) Applicant: EDWARDS LIFESCIENCES CORPORATION, Irvine, CA (US)

(72) Inventors: Tiffany Diemtrinh Tran, Anaheim, CA (US); Timothy C. Ulrich, Irvine, CA (US); Izaak Rosen, Irvine, CA (US)

(73) Assignee: EDWARDS LIFESCIENCES CORPORATION, Irvine, CA (US)

(21) Appl. No.: 19/255,255

(22) Filed: Jun. 30, 2025

Related U.S. Application Data
(63) Continuation of application No. PCT/US2024/010357, filed on Jan. 4, 2024.
(60) Provisional application No. 63/479,474, filed on Jan. 11, 2023.

(51) Int. Cl. A61F 2/24

(52) U.S. Cl. CPC A61F 2/2433

(57) ABSTRACT

Specialized sheaths and delivery systems have been developed to facilitate the introduction of prosthetic devices through incisions in the vasculature. To perform such a procedure, the vascular access site must remain accessible while minimizing blood loss. An introducer sheath inserted through the access site has a proximal sheath hub comprising a seal assembly to prevent blood loss. The loader assemblies disclosed herein enclose the prosthetic device and the delivery system, making it easier to insert them through the seal assembly of the sheath hub.

### CROSS-REFERENCE TO RELATED APPLICATIONS

This application is a continuation of PCT Patent Application No. PCT/US2024/010357 filed on Jan. 4, 2024, which application claims the benefit of U.S. Provisional Application No. 63/479,474, filed Jan. 11, 2023, the entire content of each of these applications being incorporated herein by this specific reference.

### FIELD

The present application is directed to a sheath for use with catheter-based technologies for repairing and/or replacing heart valves, as well as for delivering a device, such as a prosthetic valve, to a heart via the patient's vasculature.

### BACKGROUND

Endovascular delivery catheter assemblies are used to implant prosthetic devices, such as a prosthetic valve, at locations inside the body that are not readily accessible by surgery or where access without invasive surgery is desirable. For example, aortic, mitral, tricuspid, and/or pulmonary prosthetic valves can be delivered to a treatment site using minimally invasive surgical techniques.

Percutaneous interventional medical procedures utilize the large blood vessels of the body reach target destinations rather than surgically opening target site. There are many types of diseases states that can be treated via interventional methods including coronary blockages, valve replacements (TAVR) and brain aneurysms. These techniques involve using wires, catheters, balloons, electrodes and other thin devices to travel down the length of the blood vessels from the access site to the target site. The devices have a proximal end which the clinician controls outside of the body and a distal end inside the body which is responsible for treating the disease state. Percutaneous interventional procedures offer several advantages over open surgical techniques. First, they require smaller incision sites which reduces scarring and bleeding as well as infection risk. Procedures are also less traumatic to the tissue, so recovery times are reduced. Finally, interventional techniques can usually be performed much faster, and with fewer clinicians participating in the procedure, so overall costs are lowered. In some cases, the need for anesthesia is also eliminated, further speeding up the recovery process and reducing risk.

A single procedure typically uses several different guidewires, catheters, and balloons to achieve the desired effect. One at a time, each tool is inserted and then removed from the access site sequentially. For example, a guidewire is used to track to the correct location within the body. Next a balloon may be used to dilate a section of narrowed blood vessel. Last, an implant may be delivered to the target site. Because catheters are frequently inserted and removed, introducer sheaths are used to protect the local anatomy and simplify the procedure.

An introducer sheath can be used to safely introduce a delivery apparatus into a patient's vasculature (e.g., the femoral artery). Introducer sheaths are conduits that seal onto the access site blood vessel to reduce bleeding and trauma to the vessel caused by catheters with rough edges. Expandable introducer sheaths, formed of highly elastomeric materials, allow for the dilating of the vessel to be performed by the passing prosthetic device. Once the introducer sheath is positioned within the vasculature, the shaft of the delivery apparatus is advanced through the sheath and into the vasculature, carrying the prosthetic device.

An introducer sheath generally has an elongated sleeve that is inserted into the vasculature and a sheath hub that contains a sealing assembly. The sealing assembly has one or more sealing valves that allow a delivery apparatus to be placed in fluid communication with the vasculature with minimal blood loss. However, it can be difficult to advance a prosthetic device and delivery system through the sealing assembly without damaging the prosthetic device or kinking the catheters. Accordingly, there remains a need for devices, systems, and methods to facilitate the movement of the prosthetic device through the sealing assembly of the sheath hub.

### SUMMARY

An assembly for delivering a prosthetic implant is disclosed herein. The assembly includes a sheath hub including a proximal sleeve and an interior seal assembly. The interior seal assembly can include one or more seals, such as a proximal seal, an intermediate seal, and a distal seal. The interior seal assembly reversibly closes a hub lumen that extends from a proximal end of the sheath hub to a distal end of the sheath hub. A sheath is coupled to and extends distally from the sheath hub.

The assembly further includes a loader for facilitating the movement of a prosthetic device, such as a heart valve, through the sealing assembly of the sheath hub. The loader includes a base, a tube extending distally from the base and comprising a different material than the base, and a loader lumen extending through the base and the tube. The base of the loader includes a proximal region, a central region, and a distal region. The distal region has an outer diameter narrower than an outer diameter of the central region. A step defines the transition between the central region and the distal region. The distal region of the base slides into the proximal sleeve of the sheath hub to engage in a press-fit configuration. When the distal region of the base is engaged with the proximal sleeve, the tube of the loader opens the interior seal assembly of the sheath hub.

In some aspects, the loader further comprises a removably attachable loader cap. The loader cap can include a central seal. A delivery system (for example, a catheter of a delivery system) can be inserted through the central seal of the loader cap. In some aspects, the loader cap comprises a threaded surface to engage with threading on a proximal region of the base. In some aspects, the threading on the base is exterior threading and the threaded surface of the loader cap is a threaded inner surface.

The loader lumen is sized and configured to house a prosthetic heart valve. In some configurations, the assembly does include a prosthetic heart valve housed within the loader lumen. In some aspects, an inner diameter of the base widens approaching a proximal end of the base to facilitate insertion of the heart valve into the loader lumen.

As mentioned above, the base of the loader includes a proximal region, a central region, and a distal region. A step defines the transition between the central region and the distal region. In some aspects, the distal region of the base can measure from about 2 millimeters to about 20 millimeters axially between the step and a distal end of the base. In some aspects, the outer diameter of the distal region proximate the step is from about 0.3 millimeters to about 6 millimeters smaller than the outer diameter of the central region proximate the step.

The outer diameter of the distal region of the base can be slightly larger than the inner diameter of the proximal sleeve of the sheath hub, thereby creating friction between the surfaces in the press-fit configuration. In some aspects, the proximal sleeve of the sheath hub has a generally cylindrical inner surface and the distal region of the base has a generally cylindrical outer surface.

The loader includes a loader tube that extends distally from the loader base. The tube, or at least a portion of the tube, is coaxially positioned within the base of the loader. In this way, the loader tube defines at least a portion of the overall loader lumen. The loader tube can have a narrower outer diameter than the outer diameter of the loader base at the distal end of the loader base. The material of the loader tube is a lower durometer than a material of the loader base, but strong enough to withstand insertion through the interior seal assembly of the sheath hub. When the distal region of the base is engaged with the proximal sleeve in the press-fit configuration, the loader tube extends no further than a distal end of the sheath hub.

Methods of delivering prosthetic devices are also disclosed herein. Initial method steps include positioning and stabilizing a sheath within a target location, such as a vascular access site. When the sheath is positioned, the sheath hub that is coupled to the sheath is adjacent to the target location. In some aspects, initial method steps can include mounting a prosthetic implant, such as a heart valve, to the distal region of a catheter of the delivery system.

The method further includes assembling the loader. Assembling the loader includes inserting the catheter through a loader cap. For example, the catheter can be inserted through the central seal of the loader cap. Mounting a prosthetic implant to the catheter can occur before the catheter is inserted through the loader cap or after the catheter is inserted through the loader cap. The methods further include positioning the loader cap adjacent to the prosthetic device on a distal region of the catheter. The loader cap can be positioned, for example, by sliding the loader cap along the catheter until it is adjacent to the prosthetic device.

The methods can further include inserting the prosthetic device (which is mounted on the distal region of the catheter) into a proximal end of a base of a loader. The proximal end of the base can then be fastened to the loader cap to enclose the prosthetic device within the loader lumen. In some aspects, the proximal end of the base can be fastened to the loader cap by screwing the proximal end of the base to a threaded surface on the loader cap.

The method further includes inserting a distally positioned tube of the loader into a proximal sleeve of the sheath hub, pushing the tube of the loader through an interior seal assembly of the sheath hub, and pushing a distal region of the base into the proximal sleeve of the sheath hub until the proximal sleeve abuts a step on an outer surface of the base, thereby rotationally and axially securing the base to the sheath hub. Finally, the catheter and the prosthetic device are pushed through the loader lumen and through the sheath.

Various aspects of the implementations described above can be combined based on desired sheath system characteristics.

### DESCRIPTION OF DRAWINGS

FIG. 1 is an elevation view of an expandable sheath along with an endovascular delivery apparatus for implanting a prosthetic implant.

FIG. 2 is an elevation view of an expandable sheath including an introducer hub, a sheath locking sleeve, and an introducer.

FIG. 3 is an elevation view of the expandable sheath of FIG. 2 along with an endovascular delivery apparatus for implanting a prosthetic implant.

FIG. 4 is an elevation view of an expandable sheath a sheath hub, an introducer hub, and a sheath locking sleeve of FIG. 2.

FIG. 5A is a cross section view of the sheath hub, introducer hub, and sheath locking sleeve of FIG. 2.

FIG. 5B is a cross section view of the introducer cap, the sheath hub, the introducer hub, the sheath locking sleeve of FIG. 2.

FIG. 6 is a cross section view of the introducer cap, sheath hub, introducer hub, and sheath locking sleeve of FIG. 2.

FIG. 7 is a distal end view of the sheath locking sleeve of FIG. 2 and the proximal fluid seal of FIGS. 5A-B.

FIG. 8A is a first elevation view of the introducer hub of FIG. 2 coupled to an introducer.

FIG. 8B is a second (rotated) elevation view of the introducer hub of FIG. 2 coupled to the introducer.

FIG. 8C is a distal end view of the introducer hub of FIG. 2 coupled to the introducer.

FIG. 8D is a partial side view of the introducer hub of FIG. 2 coupled to the introducer.

FIG. 8E is a partial perspective view of the introducer hub of FIG. 2 coupled to the introducer.

FIG. 8F is a partial perspective view of the introducer hub of FIG. 2 coupled to the introducer.

FIG. 9A is a distal end view of the introducer hub of FIG. 2.

FIG. 9B is a first elevation view of the introducer hub of FIG. 2.

FIG. 9C is a proximal end view of the introducer hub of FIG. 2.

FIG. 9D is a first perspective view of the introducer hub of FIG. 2.

FIG. 9E is a second elevation view of the introducer hub of FIG. 2.

FIG. 9F is a second perspective view of the introducer hub of FIG. 2.

FIG. 10A is a distal end view of the sheath locking sleeve of FIG. 2.

FIG. 10B is a first elevation view of the sheath locking sleeve of FIG. 2.

FIG. 10C is a proximal end view of the sheath locking sleeve of FIG. 2.

FIG. 10D is a first perspective view of the sheath locking sleeve of FIG. 2.

FIG. 10E is a second elevation view of the sheath locking sleeve of FIG. 2.

FIG. 10F is a second perspective view of the sheath locking sleeve of FIG. 2.

FIG. 11 is a side elevation cross section view of a portion of the expandable sheath of FIGS. 1 and 2.

FIG. 12 is a magnified view of a portion of the expandable sheath of FIGS. 1 and 2.

FIG. 13A is a magnified view of a portion of the expandable sheath of FIGS. 1 and 2 with the outer layer removed for purposes of illustration.

FIG. 13B is a magnified view of a portion of the braided layer of the sheath of FIGS. 1 and 2.

FIG. 14 is a magnified view of a portion of the expandable sheath of FIGS. 1 and 2 illustrating expansion of the sheath as a prosthetic device is advanced through the sheath.

FIG. 15 is a side view of the expandable sheath of FIGS. 1 and 2.

FIG. 16 is a magnified section view of the sheath of FIG. 15 along section line 16-16.

FIG. 17 is cross section view of the unexpanded sheath of FIG. 16 along section line 17-17.

FIG. 18 is cross section view of the unexpanded sheath of FIG. 15 along section line 18-18.

FIG. 19 is cross section view of the unexpanded sheath of FIG. 15 along section line 19-19.

FIG. 20 is cross section view of the expanded sheath of FIG. 15 along section line 19-19.

FIG. 21 is a side view of the expandable sheath of FIGS. 1 and 2.

FIG. 22 is a cross section view of the unexpanded sheath of FIG. 21 along section line 22-22.

FIG. 23 is a cross section view of the expanded sheath of FIG. 21 along section line 22-22.

FIG. 24 is an exploded perspective view of a loader assembly used for loading the balloon catheter and prosthetic valve into the introducer sheath assembly.

FIGS. 25A and 25B are side views illustrating the insertion of the delivery system into the loader assembly.

FIG. 26 is a side view illustrating the relationship between the delivery system, the introducer sheath assembly, and the loader assembly.

### DETAILED DESCRIPTION

The following description of certain examples of the inventive concepts should not be used to limit the scope of the claims. Other examples, features, aspects, implementations, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

For purposes of this description, certain aspects, advantages, and novel features of the aspects of this disclosure are described herein. The described methods, systems, and apparatus should not be construed as limiting in any way. Instead, the present disclosure is directed toward all novel and nonobvious features and aspects of the various disclosed aspects, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Features, integers, characteristics, compounds, chemical moieties, or groups described in conjunction with a particular aspect or example of the present disclosure are to be understood to be applicable to any other aspect or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The present disclosure is not restricted to the details of any foregoing aspects. The present disclosure extends to any novel one, or any novel combination, of the features disclosed in this specification (including any accompanying claims, abstract, and drawings), or to any novel one, or any novel combination, of the steps of any method or process so disclosed.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

The terms “proximal” and “distal” as used herein refer to regions of a sheath, catheter, or delivery assembly. “Proximal” means that region closest to handle of the device, while “distal” means that region farthest away from the handle of the device.

“Axially” or “axial” as used herein refers to a direction along the longitudinal axis of the sheath.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

Disclosed aspects of an expandable sheath can minimize trauma to the vessel by allowing for temporary expansion of a portion of the introducer sheath to accommodate the delivery system, followed by a return to the original diameter once the device passes through. Disclosed aspects of the introducer sheath prevent the introducer from separating from the sheath during insertion by locking of the proximal hub of the introducer to the proximal hub of the sheath. Fixing the introducer and the sheath prevents the introducer from moving backward during insertion, thereby maintaining a snug fit and smooth transition between the introducer and the distal end of the sheath. Furthermore, present aspects can reduce the length of time a procedure takes, as well as reduce the risk of a longitudinal or radial vessel tear, or plaque dislodgement because only one sheath is required, rather than several different sizes of sheaths. Aspects of the present expandable sheath can avoid the need for multiple insertions for the dilation of the vessel.

Expandable introducer sheaths are disclosed in U.S. Pat. No. 8,690,936, entitled “Expandable Sheath for Introducing an Endovascular Delivery Device into a Body,” U.S. Pat. No. 8,790,387, entitled “Expandable Sheath for Introducing an Endovascular Delivery Device into a Body,” U.S. Pat. No. 10,639,152, entitled “Expandable Sheath and Methods of Using the Same,” U.S. Pat. No. 10,792,471, entitled “Expandable Sheath,” U.S. Patent No. application Ser. No. 16/407,057, entitled “Expandable Sheath with Elastomeric Cross Sectional Portions,” U.S. Pat. No. 10,327,896, entitled “Expandable Sheath with Elastomeric Cross Sectional Portions,” U.S. Pat. No. 11,273,062, entitled “Expandable Sheath,” Application No. PCT/US2021/019514, entitled “Expandable sheath for introducing an endovascular delivery device in to a body,” Application No. PCT/US2021/031227, entitled “Expandable sheath for introducing an endovascular delivery device into a body,” Application No. PCT/US2021/031275, entitled “Expandable sheath for introducing an endovascular delivery device into a body,” U.S. application Ser. No. 17/113,268, entitled “Expandable Sheath and Method of Using the Same,” Application No. PCT/US2021/058247, entitled “Self-Expanding, Two Component Sheath,” Application No. PCT/US2022/012785, entitled “Expandable Sheath,” U.S. Pat. No. 11,051,939, entitled “Active Introducer Sheath System,” Application No. PCT/US2022/012684, entitled “Introducer with Sheath Tip Expander,” U.S. application Ser. No. 17/078,556, entitled “Advanced Sheath Patterns,” Application No. PCT/US2021/025038, entitled “Low temperature hydrophilic adhesive for use in expandable sheath for introducing an endovascular delivery device into a body,” Application No. PCT/US2021/050006, entitled “Expandable Sheath Including Reversable Bayonet Locking Hub,” U.S. Provisional Application No. 63/280,251, entitled “Expandable Sheath Gasket to Provide Hemostasis,” the disclosures of which are herein incorporated by reference.

Disclosed herein are elongate introducer sheaths that are particularly suitable for delivery of implants in the form of implantable heart valves, such as balloon-expandable implantable heart valves. Balloon-expandable implantable heart valves are well-known and will not be described in detail here. An example of such an implantable heart valve is described in U.S. Pat. No. 5,411,552, and also in U.S. Pat. No. 9,393,110, both of which are hereby incorporated by reference. The expandable introducer sheaths disclosed herein may also be used to deliver other types of implantable medical device, such as self-expanding and mechanically expanding implantable heart valves, stents or filters. Beyond transcatheter heart valves, the introducer sheath system can be useful for other types of minimally invasive surgery, such as any surgery requiring introduction of an apparatus into a subject's vessel. For example, the introducer sheath system can be used to introduce other types of delivery apparatus for placing various types of intraluminal devices (e.g., stents, stented grafts, balloon catheters for angioplasty procedures, etc.) into many types of vascular and non-vascular body lumens (e.g., veins, arteries, esophagus, ducts of the biliary tree, intestine, urethra, fallopian tube, other endocrine or exocrine ducts, etc.). The term “implantable” as used herein is broadly defined to mean anything—prosthetic or not—that is delivered to a site within a body. A diagnostic device, for example, may be an implantable.

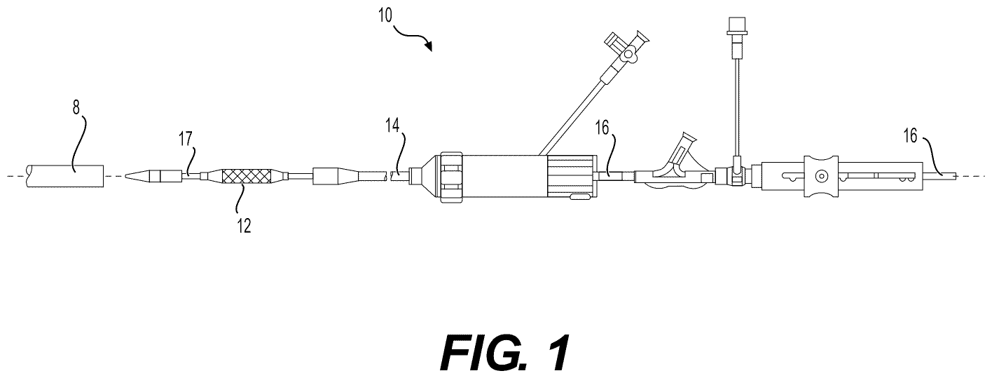

FIG. 1 illustrates an exemplary sheath 8 in use with a representative delivery system 10, for delivering a prosthetic device (implant 12), or other type of implantable (e.g., tissue heart valve), to a patient. The delivery system 10 can include a steerable guide catheter 14 (also referred to as a flex catheter) and a balloon catheter 16 extending through the guide catheter 14, and a nose catheter 17 extending through the balloon catheter 16. The guide catheter 14, balloon catheter 16, and nose catheter 17 in the illustrated example are adapted to slide longitudinally relative to each other to facilitate delivery and positioning of the prosthetic device (implant 12) at an implantation site in a patient's body as described in detail below. It is contemplated that the sheath 8 can be used with any type of elongated delivery apparatus used for implanting balloon-expandable prosthetic valves, self-expanding prosthetic valves, and other prosthetic devices.

As described in more detail below, in general, the sheath 8 comprises an elongate expandable tube that, in use, is inserted into a vessel (e.g., transfemoral vessel, femoral artery, iliac artery) by passing through the skin of patient, such that the distal end of the sheath 8 is inserted into the vessel. Sheath 8 includes a hemostasis valve and/or sealing features at the proximal end of the sheath, e.g., in the sheath hub 20, that provide hemostasis and prevents blood leakage from the patient through the sheath 8. The sheath 8, including an introducer 6, is advanced into the patient's vasculature. Once positioned the introducer 6 is removed and the delivery system 10 is inserted into/through the sheath 8, and the prosthetic device (implant 12) then be delivered and implanted within patient.

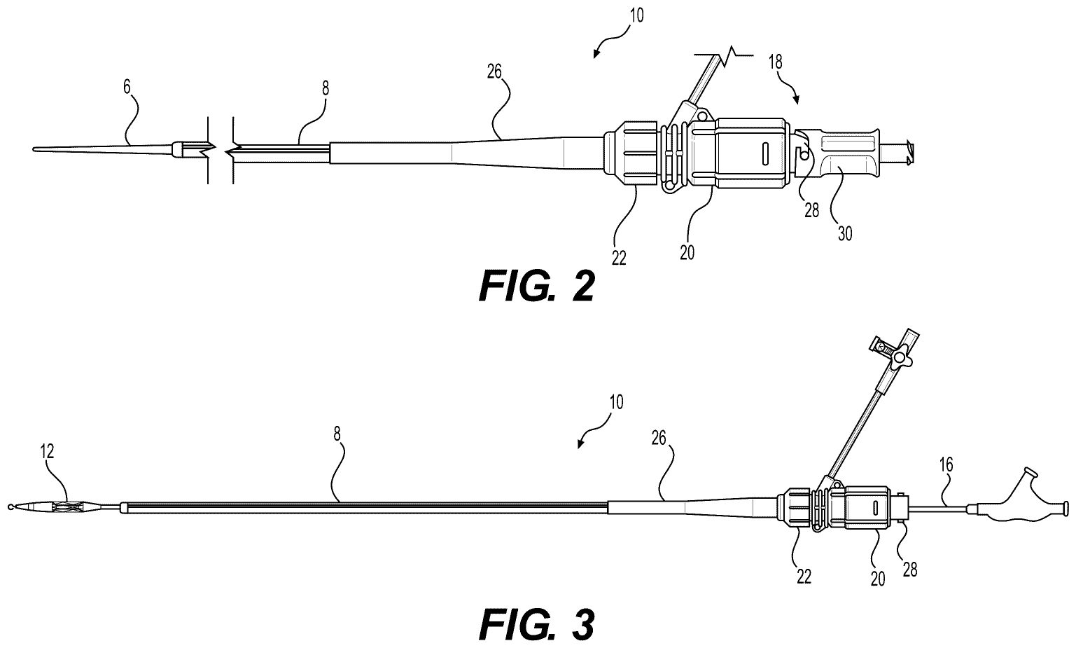

FIGS. 2 and 3, the introducer device/sheath assembly includes a sheath hub 20 at a proximal end of the device and an expandable sheath 8 extending distally from the sheath hub 20. The sheath 8 is coupled to the sheath hub 20 which in turn is removably coupled to a sheath locking system 18. The sheath locking system 18 allows the introducer 6, or other device desired to be removably coupled (axially and rotatably) to the sheath 8.

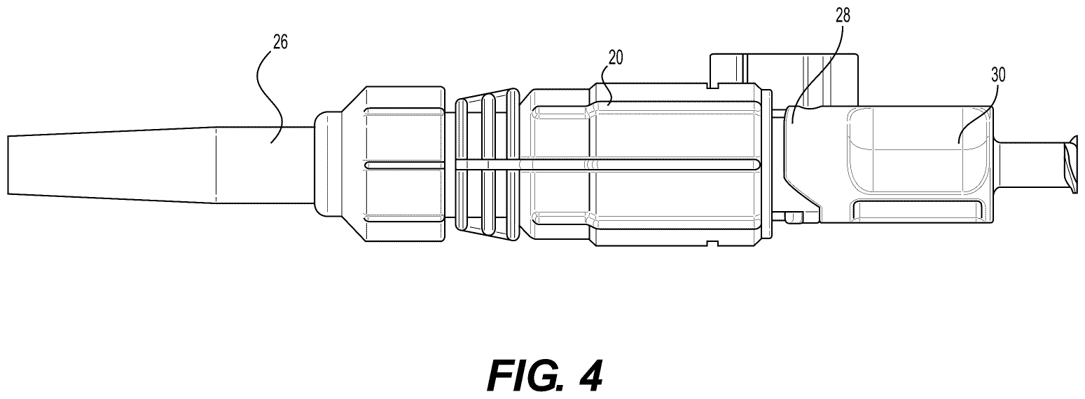

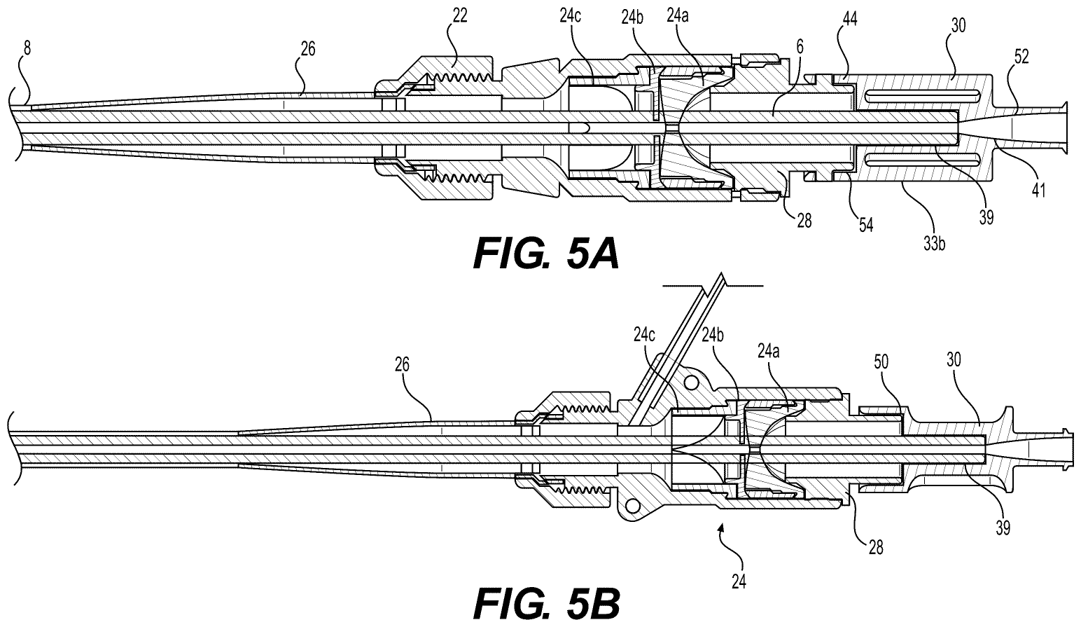

As illustrated in FIGS. 2-6, the sheath hub 20 can function as a handle for the device. Sheath hub 20 also provides a housing for necessary seal assemblies and an access point for a secondary lumen (e.g., fluid lumen) in fluid communication with the central lumen of the sheath hub 20. The seal assembly 24, as described above and as shown in FIGS. 5A and 5B, is included in the sheath hub 20. The seal assembly 24 includes a proximal seal 24a, an intermediate seal 24b, and a distal seal 24c. When assembled, the introducer 6 passes through the seal assembly and extends distal of the sheath 8. The proximal seal 24a, the intermediate seal 24b, and the distal seal 24c are each formed to prevent unwanted fluid from advancing in the proximal direction through the sheath hub 20 and proximal of the seal assembly 24. They are each openable and closable to provide pressure variation to affect the desired fluid flow from a physician or technician.

The distal end of the sheath hub 20 includes threads 21 for coupling to a threaded sheath hub cap 22. The sheath 8 is provided between the sheath hub 20 and the sheath hub cap 22 such that coupling the sheath hub cap 22 to the sheath hub 20 fixes the sheath 8 to the sheath hub 20. The sheath hub cap 22 is a cylindrical cap having a cap body having a proximal end and a distal end and defining a central lumen extending longitudinally between the proximal end and the distal end. The sheath hub cap 22 has a larger diameter at its proximal end than at its distal end.

The sheath hub 20 further has receiving slots 48 for coupling the sheath locking system 18, particularly the sheath locking sleeve 28, to the sheath hub 20. The receiving slots 48 are openings which extend around a portion of the diameter of the sheath hub 20 and are sized and configured to accept the interface diameters 66 of the sheath locking sleeve 28. Coupling between the receiving slots 48 and the interface diameters 66 axially and rotationally fixes the sheath locking sleeve 28 and the sheath hub 20 relative to each other.

FIG. 2 illustrates the sheath 8 of FIG. 1 including a sheath locking system 18 which prevents axial and rotational translation of the introducer 6 with respect to the sheath 8. Example locking systems are disclosed in PCT/US2021/050006, entitled “Expandable Sheath Including Reverse Bayonet Locking Hub,” the disclosure of which is incorporated herein by reference. It is contemplated that the locking system disclosed herein can also be used to couple the sheath 8/sheath hub 20 with other delivery system components, catheters, dilators, etc. including the same mating features.

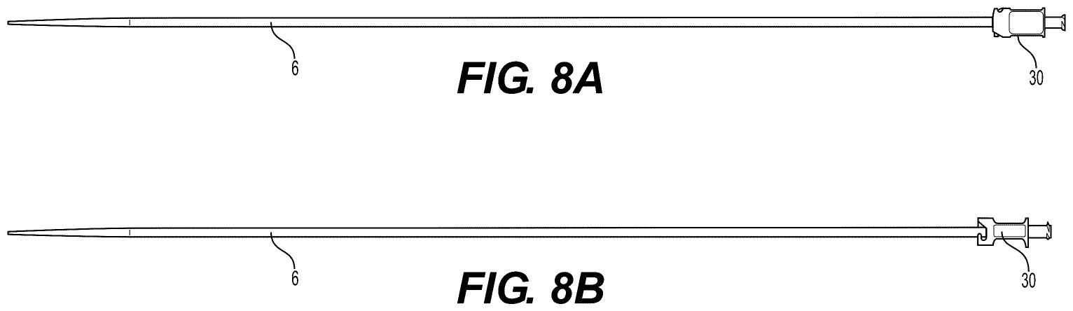

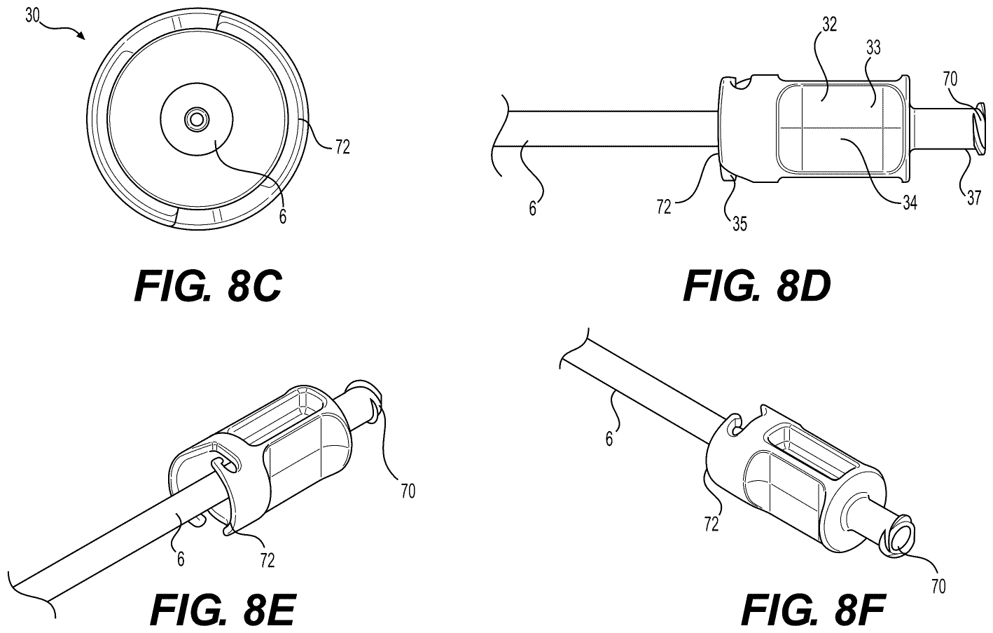

The sheath locking system 18 keeps the introducer 6 fixed with respect to the sheath 8 during insertion without requiring a physician or technician to hold the introducer 6 and the sheath 8 in place at the distal end. As illustrated in FIGS. 8A-8B, the sheath locking system 18 includes a sheath locking sleeve 28 and an introducer hub 30 (including corresponding introducer 6). The sheath locking sleeve 28 is coupled to the sheath 8 via the sheath hub 20. The sheath locking sleeve 28 engages the introducer hub 30 and is moveable between a locked and unlocked position, thereby fixing the position of the introducer 6 and the sheath 8 and preventing movement therebetween, particularly during insertion into the patient. As will be described in more detail below, the sheath locking system 18 keeps the introducer 6 from separating from the sheath 8 and prevents gaps from forming that can cause patient abrasions and unintended fluid flow between the introducer 6 and the sheath 8 during insertion.

FIGS. 2, 5A-5B and 6, and illustrate the sheath locking sleeve 28 coupled to the introducer hub 30 and the sheath hub 20. As will be described in more detail below, the sheath locking sleeve 28 includes a guide 31 that engages a locking channel 38 provided on the introducer hub 30. The guide 31 moves within the locking channel 38 between an unlocked position, where the sheath locking sleeve 28 is rotationally and axially movable with respect to the introducer hub 30, and a locked position (FIG. 2), where the sheath locking sleeve 28 is axially fixed with respect to the introducer hub 30.

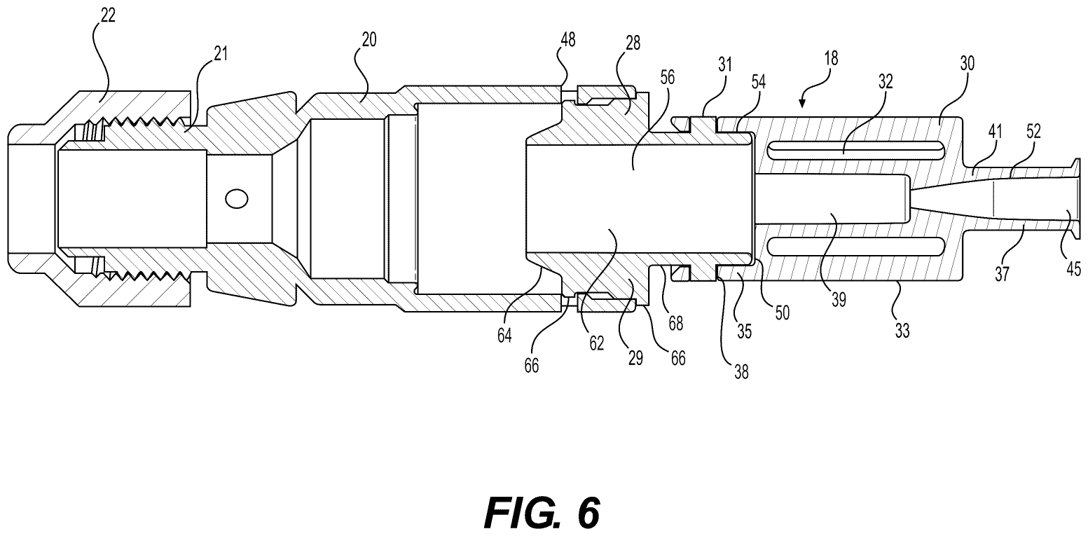

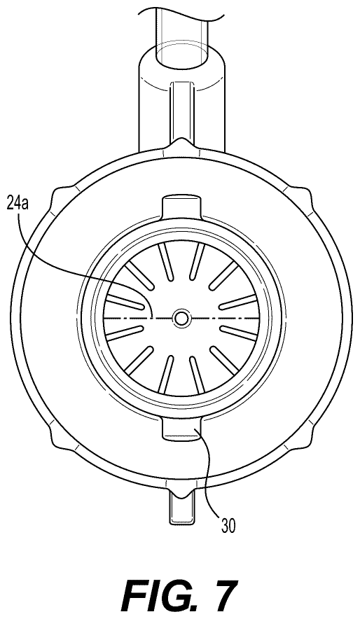

The sheath locking sleeve 28 is illustrated, for example, in FIGS. 10A-10F. The sheath locking sleeve 28 includes an elongated sleeve body 29 with a central lumen 56 extending longitudinally between the proximal end 58 and distal end 60 of the sleeve body 29. As provided in FIG. 6, the central lumen 56 defines a generally cylindrical inner surface 62 of the sheath locking sleeve 28. The central lumen 56 has a diameter of at least 0.3″. In some examples, the diameter ranges between 0.3″ and 0.6″. Preferably, the diameter is about 0.40″. The distal end 60 of the sleeve body 29 also has a frustoconical outer surface 64 that tapers about the distal end 60 to help with positioning the sheath locking sleeve 28 within the sheath hub 20 and abutting the seal assembly 24 (FIGS. 5B and 5B). The sheath locking sleeve 28 also has a plurality of interface diameters 66 that extend radially from the outer surface of the sleeve body 29 around (all or a portion of) the circumference of the sheath locking sleeve 28. As illustrated in FIG. 5A and 6, the distal interface diameters 66 are sized and configured to engage corresponding recesses and/or receiving slots 48 provided in the sheath hub 20 for securing the sheath locking sleeve 28 to the sheath hub 20, and the distal interference diameter 66 seat against the proximal end of the sheath hub 20.

The sheath locking sleeve 28 includes a guide 31 projecting from the outer surface 68 of the sheath locking sleeve 28. The guide 31 engages a corresponding shaped locking channel 38 in the introducer hub 30. The guide 31 extends radially from the outer surface 68 and at least partially around the circumference of the outer surface 68. As provided in FIG. 6, the top surface of the guide 31 does not extend beyond the outer surface of the introducer hub 30 when the sheath locking sleeve 28 and the introducer hub 30 are coupled. For example, the height of the guide 31 corresponds to the wall thickness of the introducer hub 30 proximate the guide when the sheath locking sleeve 28 and the introducer hub 30 are coupled. In another example, the top surface of the guide 31 is recessed with respect to the outer surface of the introducer hub 30. That is, the height of the guide 31 is less than the wall thickness of the introducer hub 30. In other examples, the height of the guide 31 is greater than a wall thickness of the introducer hub 30 such that the top surface of the guide 31 extends beyond the outer surface of the introducer hub 30 when the sheath locking sleeve 28 and the introducer hub 30 are coupled. In some examples, the height/axial length of the guide 31 is between about 0.050″ and about 0.10.″ In some examples that height/axial length of the guide 31 is about 0.075″.

As illustrated in FIGS. 10D-10F, the guide 31 is a cylindrically shaped projection. However, it is contemplated that the guide 31 may have any other regular or irregular shape that would facilitate movement of the guide 31 within the locking channel 38 of the introducer hub 30. For example, the guide 31 may have an elongated hexagon shape. The guide 31 can have a diameter/width ranging from about 0.05″ to about 0.20″. Preferably the guide 31 has a diameter/width of about 0.100″.

In general, the sheath locking sleeve 28 can be formed from polycarbonate, but in other aspects, the sheath locking sleeve 28 can be formed from rigid plastic, or any other material suitable for providing a strong locking connector for an introducer 6 (metal, composite, etc.)

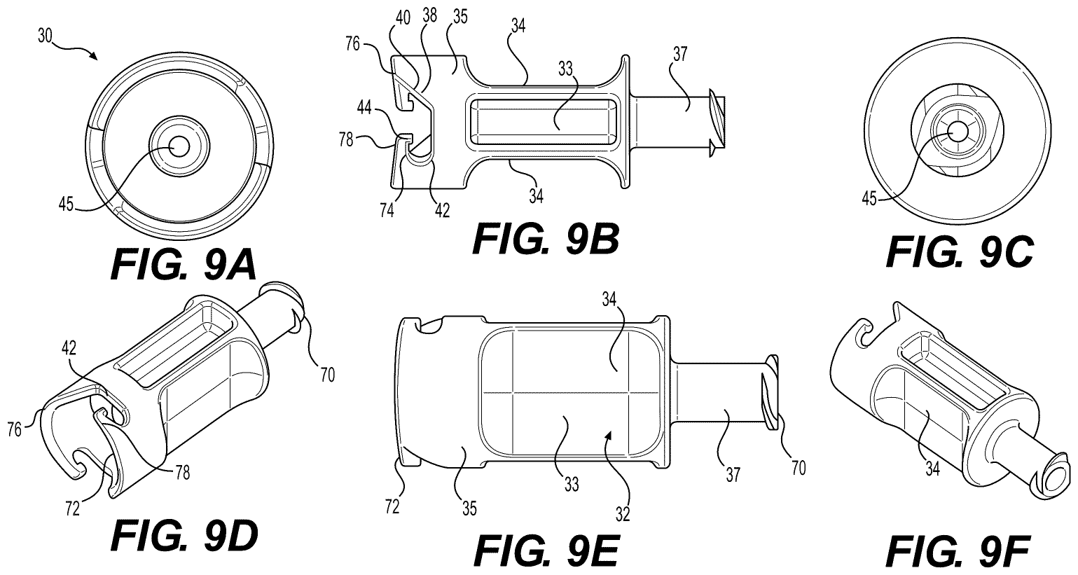

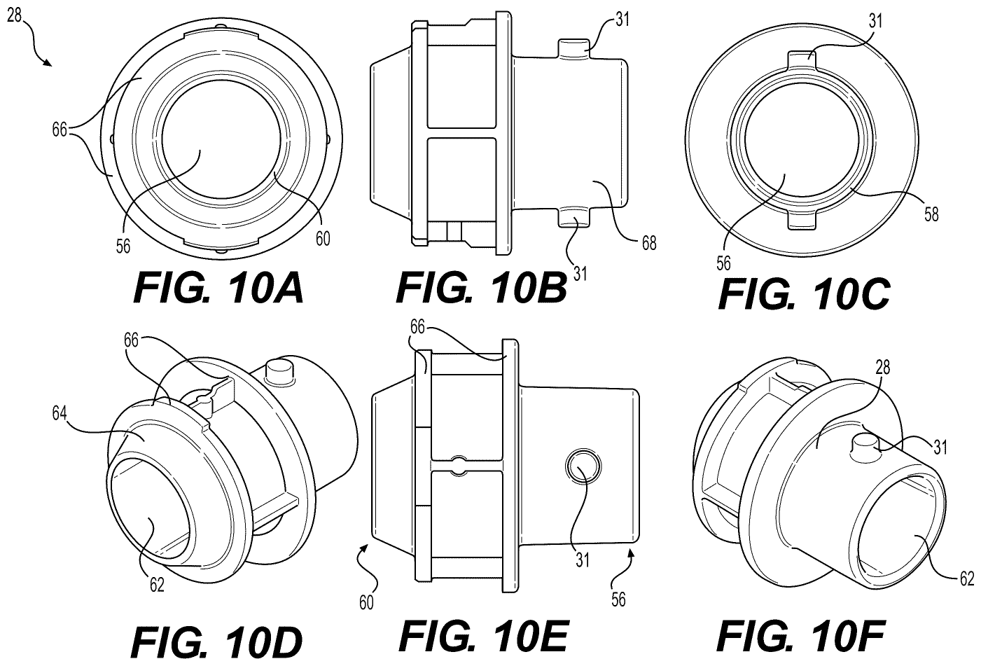

FIGS. 2-6 illustrate the introducer hub 30 coupled to the sheath locking sleeve 28. FIGS. 8A-8F show the introducer hub 30 coupled to the introducer 6. FIGS. 9A-9F provide multiple view of the introducer hub 30. As described herein, the introducer 6 is fixedly coupled to the introducer hub 30. The introducer hub 30 couples with the sheath locking sleeve 28 to fix the position the introducer 6 (axially and rotationally) with respect to the sheath locking sleeve 28/sheath 8. Each of the introducer 6 and introducer hub 30 are described in more detail as follows.

FIGS. 8A-8F illustrate the introducer hub 30 with the introducer 6 coupled thereto. Example introducer sheaths are described, for example in U.S. Pat. Nos. 8,690,936 and 8,790,387, the disclosures of which are incorporated herein by reference. As provided in the cross-section views of FIGS. 5A and 5B, the introducer 6 is coupled to the introducer hub 30 and extends beyond the distal end of the introducer hub 30 body and into the sheath 8. When coupled to the sheath hub 20, the introducer 6 extends through the central lumen 56 of the sheath locking sleeve 28, the sheath hub 20 and the central lumen of the sheath 8. As will be descried below, the sheath 8 generally comprises a radially expandable tubular structure. Passage of the introducer 6 through the sheath 8 and into a patient's vasculature causes the blood vessel to radially expand to about the diameter of the sheath 8. That is, the diameter of the central lumen of the sheath 8 is generally abuts the outer diameter of the introducer 6 such that the introducer 6 provides a mechanism to expand a patient's vessel to accept the sheath.

As provided in FIGS. 8A-8F, the introducer 6 is formed as an elongate body with a central lumen extending therethrough. As shown in FIGS. 5A and 5B, the central lumen of the introducer is aligned with the central lumens of the introducer hub 30, the sheath hub 20 and the sheath 8. The introducer 6 is received within a recessed opening 39 provided on an interior surface of the introducer hub 30, the recessed opening 39 axially aligned with the central lumen 45 of the introducer hub 30. The introducer 6 is coupled to the introducer hub 30 at the recessed opening 39. In an example system, the introducer 6 has a diameter corresponding to, or less than, the diameter of the recessed opening 39. In some examples, the introducer 6 is fixedly coupled to the introducer hub 30 at the recessed opening 39. For example, the introducer 6 is coupled to the recessed opening 39 of the introducer hub 30 by at least one of a press fit, an interference fit, a snap fit, a mechanical fastener, a chemical fastener (e.g., an adhesive), a weld, a thermal process, and/or any other suitable coupling process known in the art.

As described above, the introducer 6 has a central lumen that aligns with the central lumen 45 of the introducer hub 30. This joined lumen allows for the passage of surgical equipment and/or medical devices to the treatment site (e.g., a guide wire). In an example system, and as provided in FIGS. 5A and 5B, the central lumen of the introducer 6 has a diameter corresponding to at least a portion of the diameter of the central lumen 45 of the introducer hub 30. In general, the corresponding diameter portion is adjacent the distal end of the central lumen 45. In other examples, the diameter of the central lumen 45 at the distal end of the introducer hub 30 is slightly larger than the diameter of the central lumen passing through the introducer 6. The central lumen 45 can also define a decreasing tapered portion 41 between the proximal end and the distal end of the introducer hub 30 (see FIG. 6). The corresponding diameter portion and decreasing tapered portion 41 allows for smooth transition and delivery of surgical equipment and/or medical device through the introducer hub 30 and into the central lumen of the introducer 6.

As illustrated in FIGS. 9A-9F, the introducer hub 30 includes a hub body 32 having a proximal end 70 and a distal end 72 and defining a central lumen 45 extending therethrough. The hub body 32 has a first (middle) portion 33, a second (distal) portion 35 which extends distally from the first portion 33 and a third (proximal) portion 37 which extends proximally from the first portion 33. The first portion 33 includes the cylindrically-shaped recessed opening 39 for receiving and retaining the introducer 6 and an outer surface 33b. In some examples, the recessed opening 39 has a diameter ranging between 0.15″ and about 0.25″. In some examples, the recessed opening 39 has a diameter ranging between 0.17″ and about 0.20″. In some examples, the recessed opening has a diameter of about 0.194″.

The third (proximal) portion 37 of the introducer hub 30 includes the decreasing tapered portion 41 of the central lumen 45. The decreasing tapered portion 41 defining a frustoconical shape with decreasing taper/diameter from the proximal to the distal end of the sheath. It is contemplated that the tapered portion 41 has a minimum diameter of about 0.007″ and a maximum diameter of about 0.194″.

As illustrated in FIG. 5A and B, when coupled, the central lumen 56 of the sheath locking sleeve 28 is aligned with the central lumen 45 of the introducer hub 30. In some examples, the central lumen 56 of the sheath locking sleeve 28 is coaxial with the central lumen 45 of the introducer hub 30. When coupled, the proximal end of the sheath locking sleeve 28 is received within the central lumen 45 of the introducer hub 30. The proximal end surface of the sheath locking sleeve 28 is adjacent a shoulder 50 provided on an inner surface of the central lumen 45 of the introducer hub 30. As illustrated in FIGS. 5A and 5B, the central lumen 45 of the introducer hub 30 includes a first portion 52 having a first diameter adjacent the proximal end of the introducer hub 30, and a second portion 54 having a second, larger, diameter adjacent the distal end of the introducer hub 30. The recessed opening 39 can be considered either a component of the first portion 52 of the central lumen 45, or a separate component of the central lumen 45 located between the first (proximal) portion 52 and the second (distal) portion 54. When the sheath locking sleeve 28 and introducer hub 30 are coupled, at least a portion of the sleeve body 29 of the sheath locking sleeve 28 is received within the second portion 54 (larger portion) of the central lumen 45 of the introducer hub 30. The central lumen 56 of the sheath locking sleeve 28 is aligned with the central lumen 45 of the introducer hub 30 such that they are co-axial and form a smooth inner surface along the combined central lumens of the introducer hub 30 and the sheath locking sleeve 28.

As described generally above, the sheath locking sleeve 28 couples to the introducer hub 30 via engagement between the guide 31 on the sheath locking sleeve 28 and the locking channel 38 provided in the introducer hub 30. As provided in FIGS. 9A-9F, the introducer hub 30 includes two locking channels 38. However, it is contemplated that the introducer hub 30 can include one locking channel 38 or more than two locking channels 38. The locking channel 38 can be is formed a recess or groove in a surface of the introducer hub 30, as a slotted opening, a clip, or as any other feature capable of receiving and securing the guide 31 projecting from the outer surface of the sheath locking sleeve 28 with the introducer hub 30. Illustrated in FIG. 9B, the locking channels 38 provide an interface to secure the sheath locking sleeve 28 to the introducer hub 30 and ensure a fixed axial position between the introducer 6 and the sheath 8.

The locking channel 38 is formed on the distal end of the introducer hub 30. The locking channel 38 includes an opening on the distal end surface that leads to an angled guide portion 40 that transitions to a locking portion 42. The guide portion 40 is configured to direct the guide 31 of the sheath locking sleeve 28 in an axial and circumferential direction along the side wall of the guide portion 40 towards the locking portion 42 upon rotation of the introducer hub 30 and/or the sheath locking sleeve 28. The locking portion 42 is configured to securely engage the guide 31, fixing the axial position of the introducer hub 30 with respect to the sheath locking sleeve 28. As illustrated in FIG. 9B, the guide portion 40 of the locking channel 38 extends from the distal end of the introducer hub 30 axially towards the proximal end of the introducer hub 30 and circumferentially around the introducer hub 30. For example, the guide portion 40 of the locking channel 38 can be described as extending helically around/along a length of the introducer hub 30 or on an angle from the distal end of the introducer hub 30.

As illustrated in FIGS. 9B and 9D, the locking portion 42 of the locking channel 38 extends at an angle from the end of the guide portion 40. As provided in FIG. 9B, the angle between the centerline of the guide portion 40 and the centerline of the locking portion 42 is greater than 90-degrees. In another example, the angle between the centerline of the guide portion 40 and the centerline of the locking portion 42 is about 120-degrees. In an example system, the locking portion 42 extends around a portion of the circumference of the introducer hub 30. The locking portion 42 can extend parallel to the distal end of the introducer hub 30. In an example system, the length of the guide portion 40 (measured along its centerline) is greater than a length of the locking portion 42 (measured along its centerline). In another example, the length of the guide portion 40 equals or is less than a length of the locking portion 42.

The locking portion 42 can include a catch 44 for securing the guide 31 within the locking portion 42 of the locking channel 38 and forming a partial barrier for the guide 31 within the locking portion 42. As illustrated in FIG. 9B, the catch 44 includes a projection that extends from a side wall 74 of the locking portion 42 and releasably secures the guide 31 within the locking channel 38. The catch 44 extends from the side wall 42a of the locking portion 42 in a proximal direction towards the center line of the locking portion 42 and has a height sufficient to retain the guide 31 between the catch 44 and the end of the locking portion 42.

The distal end surface (distal end 72) of the introducer hub 30 can include features for biasing the guide 31 towards the locking channel 38. For example, the distal end of the introducer hub 30 can include a tapered surface angled toward an opening of the locking channel 38. As illustrated in FIG. 9B, the distal end 72 of the introducer hub 30 includes a first tapered surface 76 (angled towards a leading edge of the opening of the locking channel 38 and a second tapered surface 78 angled towards the trailing edge of the opening of the locking channel 38.

In use, engagement between the guide 31 and the guide portion 40 of the locking channel 38 is configured to bias the sheath locking sleeve 28 in a proximal axial direction toward the proximal end 70 of the introducer hub 30 (towards a locked position) when the sheath locking sleeve 28 is rotated in a first axial direction. In this direction the guide 31 advances toward the locking portion 42 of the locking channel 38 into the locked position. Alternatively, engagement between the guide 31 and the locking portion 42 of the locking channel 38 is configured to bias the sheath locking sleeve 28 in a distal axial direction toward the distal end of the introducer hub 30 (towards an unlocked position) when the sheath locking sleeve 28 is rotated in a second (opposite) axial direction. In the second direction, the guide 31 advances away from the locking portion 42 of the locking channel 38, to the unlocked position. When the guide 31 is in the locked position and retained with by locking portion 42 by catch 44, rotation in the second direction causes the guide 31 to bias against the catch 44 overcoming the oppositional forces of the catch 44, and moving the guide 31 from the locked to the unlocked position.

As illustrated in FIGS. 8A-9F, the outer surface of the introducer hub body 32 includes gripping features and/or surfaces for a physician or technician to use when manipulating the introducer hub 30. As provided in FIG. 9B, the introducer hub body 32 can include a two recessed gripping surfaces 34 on opposite sides of the longitudinal axis of the introducer hub 30. When the introducer hub 30 is viewed from the side, the gripping surfaces 34 define a shape having a smaller diameter/width center portion and larger diameter/width end portions, e.g. dog-bone/barbell shape to the hub body 32. In an example system, the gripping surfaces 34 are provided along at least 40% of the length of the introducer hub body 32. In another example, the gripping surfaces 34 are provided along at least 50% of the length of the introducer hub body 32.

In general, the introducer hub 30 can be formed from polycarbonate, but in other aspects the introducer hub 30 can be formed from rigid plastic, or any other material suitable for providing a locking mechanism for an introducer 6 (metal, composite, etc.).

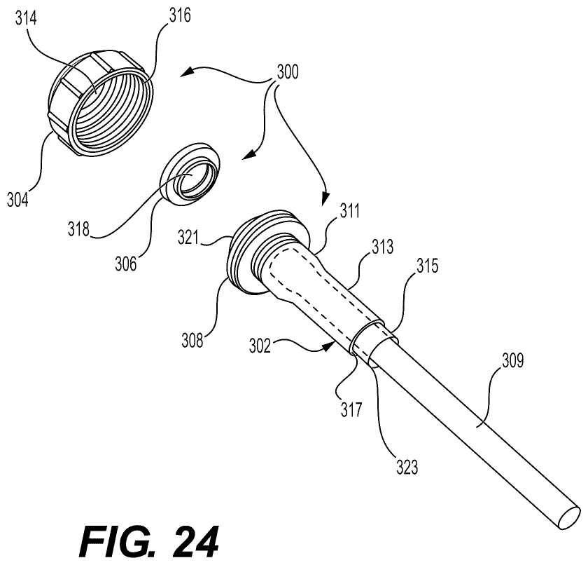

Once the sheath 8 is positioned within the patient and the introducer 6 and introducer hub 30 are withdrawn, a loader can be used to facilitate introduction of a heart valve through the sheath hub 20. Loaders are shown in FIGS. 24-26. An assembled loader encloses the flexible balloon catheter carrying the valve. The loader has a higher rigidity than the catheter, making it easier to insert the catheter and valve into the sheath hub 20. The loader also protects the valve from damage as it passes through the seal assembly 24 of the sheath hub 20. Without the loader assembly 300, it would be difficult for the delivery system 10 and the heart valve to traverse the interior seal assembly 24 without buckling or damage to the heart valve. Loaders are also described in U.S. Pat. No. 7,780,723, which is incorporated by reference herein.

With reference to FIG. 24, a loader assembly 300 includes a loader base 302, a loader cap 304, a central seal 306, and a loader tube 309 extending distally from the base 302. The loader base 302 has threading 308 for connection with the loader cap 304. The loader cap 304 includes a loader cap opening 314 in a proximal end thereof and a threaded inner surface 316 for engagement with the exterior threading 308 of the loader base 302. The central seal 306 is secured to the loader cap 304, and a seal opening 318 is aligned with the loader cap opening 314 to allow a catheter to be inserted through the cap and into the loader lumen.

Base 302 has a base lumen extending between proximal end 321 and distal end 323. As shown in FIG. 24, tube 309 is nested coaxially within the base lumen, with a portion of tube 309 extending distally outward from distal region 315 of the base 302. The outer diameter of tube 309 is narrower than the outer diameter of the distal region 315 of base 302, at least at the region where the tube 309 exits base 302. Tube 309 may extend fully through base 302, stopping just at the loader seal opening 318. Alternatively, tube 309 may stop short of loader seal opening 318. As such, tube 309 may define all of the loader lumen or part of the loader lumen. That is, the overall loader lumen can therefore be defined by the lumen of tube 309 or a combination of the lumen of base 302 and the lumen of the tube 309, depending upon how far tube 309 extends into base 302.

Tube 309 of the loader assembly 300 can be formed from a different material than base 302. For example, in some aspects, tube 309 is formed of a material having a lower durometer than base 302. Nonetheless, it will be formed of a material that can withstand insertion through seal assembly 24 of the sheath hub 20 without kinking or buckling, as discussed below. With the tube 309 fully in position within the seal assembly 24, the tube 309 extends no further than the distal end of sheath hub 20.

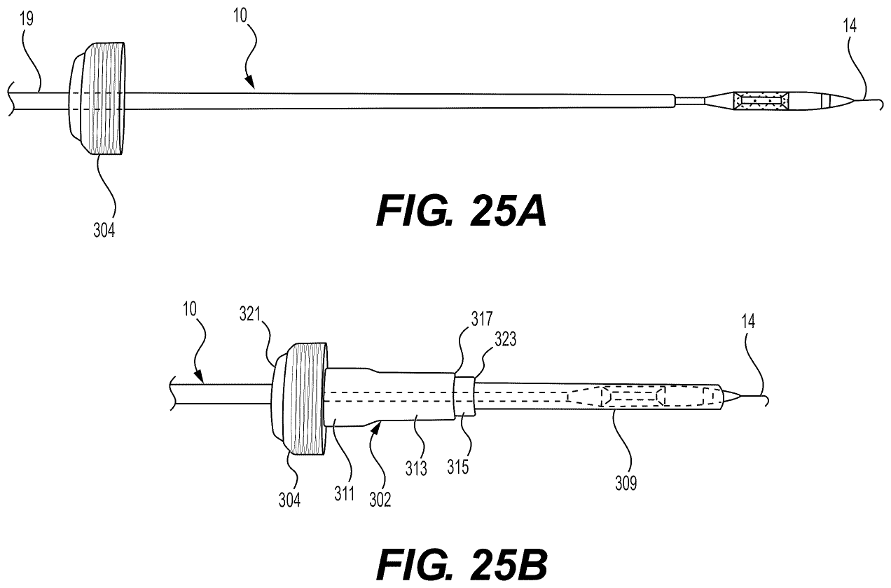

With reference to FIG. 24 and FIG. 25A, the loader cap 304 and central seal 306 are passed onto the delivery system 10 while detached from base 302. An implantable device, such as heart valve 319, is crimped onto balloon catheter 16 near the distal end of delivery system 10. The distal region of delivery system 10 (which includes the distal tip/nose catheter 17, the heart valve 319, the distal shaft of balloon catheter 16, and the balloon) is inserted into the loader lumen via proximal end 321 of the loader base 302. The loader base 302 is pulled over to cover and protect the heart valve 319 and underlying balloon. The loader base 302 comes into contact with loader cap 304, and loader cap 304 is screwed onto the threading 308 to enclose the heart valve 319 within loader lumen, as shown in FIG. 25B. Notably, the inner diameter of base 302 (and the loader lumen) can widen approaching a proximal end of the base 302. This widening of the inner diameter prevents damage or deformation to heart valve 319 during its insertion into base 302.

As seen in FIG. 25B, base 302 comprises a proximal region 311, a central region 313, and a distal region 315. The outer diameter of distal region 315 is narrower than the outer diameter of the central region 313, with step 317 defining the transition between the central region 313 and the distal region 315. In some aspects, the outer diameter of the distal region 315 proximate the step 317 is from about 0.3 millimeters to about 6 millimeters smaller than the outer diameter of the central region 313 proximate the step 317 (for example, from about 0.5 millimeters to about 4 millimeters smaller than the outer diameter of the central region 313, or from about 1 millimeter to about 2 millimeters smaller than the outer diameter of the central region 313). Distal region 315 of the base 302 measures from about 2 millimeters to about 20 millimeters axially between the step 317 and a distal end 323 of the base 302 (for example, from about 3 millimeters to about 15 millimeters between the step 317 and the distal end 323, or from about 5 to about 10 millimeters between the step 317 and the distal end 323).

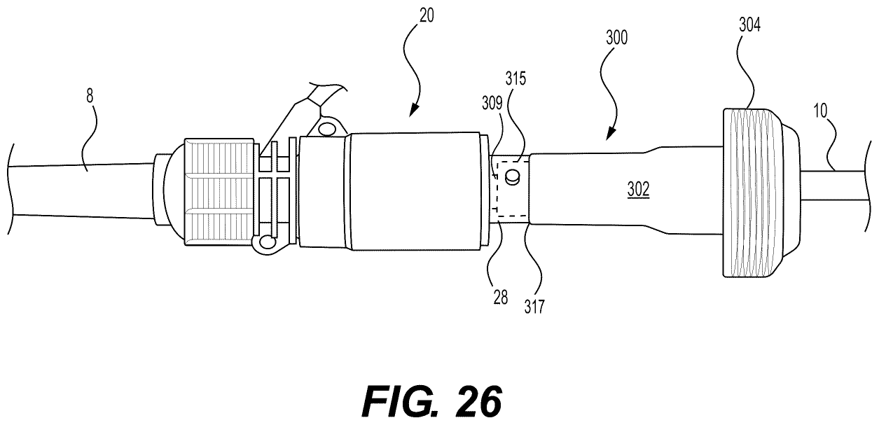

FIG. 26 shows loader base 302 assembled with sheath hub 20 in a press-fit configuration. The loader tube 309 and base 302 are inserted into the proximal sleeve 28 (i.e., the sheath locking sleeve 28) of the sheath hub 20. Distal region 315 of the base 302 slides into the proximal sleeve 28 of the sheath hub 20 until a proximal edge 27 of proximal sleeve 28 abuts step 317 of the base 302. Inner surfaces of the proximal sleeve 28 are in tight contact with outer surfaces of the distal region 315 of the loader base 302, forming a press-fit engagement that reduces rotational and axial slippage. For example, in some aspects, an outer diameter of the distal region 315 of the base 302 is slightly smaller than the inner diameter of the proximal sleeve 28 of the sheath hub 20, therefore creating friction that prevents slippage between the surfaces once engaged. This creates stability between the delivery system and the sheath hub 20, allowing users to insert the delivery system into the human body through the loader and the sheath more easily. In some aspects, the distal region 315 of the base 302 has a generally cylindrical outer surface to approximate a generally cylindrical inner surface of proximal sleeve 28. However, in some aspects, the distal region 315 of base 302 and corresponding inner surface of the proximal sleeve 28 may take non-cylindrical shapes. With the loader assembly 300 tightly engaged to the sheath hub 20, the practitioner can push the delivery system 10 and heart valve 319 into the sheath 8 with little concern that the two components will move relative to each other.

When the distal region 315 is engaged with proximal sleeve 28 (e.g., the sheath locking sleeve 28), tube 309 passes through and opens an interior seal assembly of the sheath hub 20 (similar to seal assembly 24 described above with respect to FIGS. 5A and 5B). Without the tube 309 in place, interior seal assembly 24 reversibly closes a hub lumen that extends through the sheath hub. Tube 309 of the loader assembly 300 facilitates the opening of the interior seal assembly 24 and protects heart valve 319 from damage as it traverses the sheath hub 20. With the tube 309 positioned inside the sheath hub 20, delivery system 10 is in communication with an inner passageway of the sheath 8 and thus, with the body vessel. The loader assembly 300 advantageously allows the introduction of the delivery system 10 into the sheath 8 without substantial blood loss from the patient.

As described above, the introducer device/sheath assembly includes an expandable sheath 8 extending distally from the sheath hub 20. The expandable sheath 8 has a central lumen to guide passage of the delivery system 10 for the prosthetic device (implant 12)/prosthetic heart valve. In an alternative aspect, the introducer device/sheath assembly need not include the sheath hub 20. For example, the sheath 8 can be an integral part of a component of the sheath assembly, such as the guide catheter. As described above, the sheath 8 can have a natural, unexpanded outer diameter that will expand locally upon passage of the prosthetic device (implant 12).

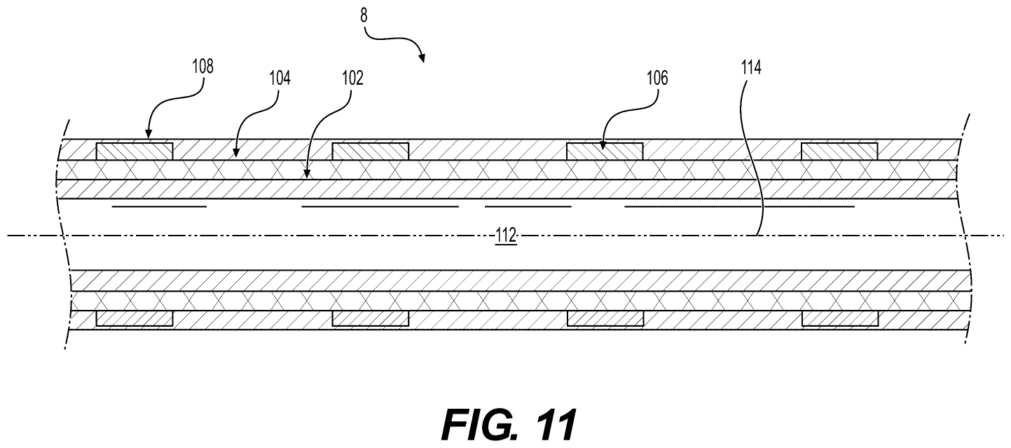

In certain aspects, the expandable sheath 8 can comprise a plurality of coaxial layers extending along at least a portion of the length of the sheath 8. The structure of the coaxial layers is described in more detail below with respect to FIGS. 11-23. Example expandable sheaths including coaxial layers are described, for example, in U.S. patent application Ser. No. 16/378,417, entitled “Expandable Sheath,” and U.S. patent application Ser. No. 17/716,882, entitled “Expandable Sheath,” the disclosures of which are herein incorporated by reference.

Various aspects of the coaxial layered structure of the sheath 8 are described herein. For example, in reference to the example sheath 8 illustrated in FIGS. 11-14, the expandable sheath 8 can include a number of layers including an inner layer 102 (also referred to as an inner layer), a second layer 104 disposed around and radially outward of the inner layer 102, a third layer 106 disposed around and radially outward of the second layer 104, and a fourth outer layer 108 (also referred to as an outer layer) disposed around and radially outward of the third layer 106. In the illustrated configuration, the inner layer 102 can define the lumen 112 of the sheath extending along a central axis 114 through which the delivery apparatus travels into the patient's vessel in order to deliver, remove, repair, and/or replace a prosthetic device, moving in a direction along the longitudinal axis of the sheath 8.

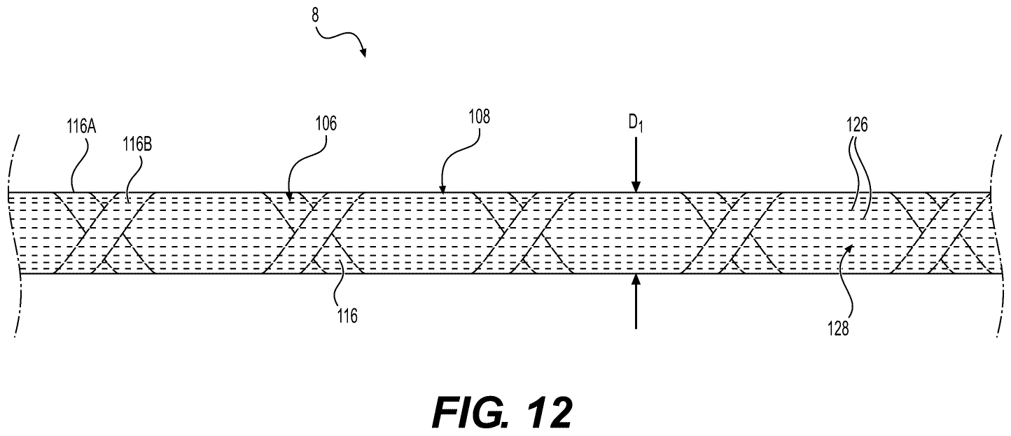

Referring to FIG. 12, when the sheath 8 is in an unexpanded state, various layers of the sheath, e.g., the inner layer 102 and/or the outer layer 108, can form longitudinally-extending folds or creases such that the surface of the sheath comprises a plurality of ridges 126 (also referred to herein as “folds”). The ridges 126 can be circumferentially spaced apart from each other by longitudinally-extending valleys 128. When the sheath expands beyond its natural diameter D₁, the ridges 126 and the valleys 128 can level out or be taken up as the surface radially expands and the circumference increases, as further described below. When the sheath 8 collapses back to its natural diameter, the ridges 126 and valleys 128 can reform.

In certain aspects, the inner layer 102 and/or the outer layer 108 can comprise a relatively thin layer of polymeric material. For example, in some aspects the thickness of the inner layer 102 can be from 0.01 mm to 0.5 mm, 0.02 mm to 0.4 mm, or 0.03 mm to 0.25 mm. In certain aspects, the thickness of the outer layer 108 can be from 0.01 mm to 0.5 mm, 0.02 mm to 0.4 mm, or 0.03 mm to 0.25 mm.

In certain examples, the inner layer 102 and/or the outer layer 108 can comprise a lubricious, low-friction, and/or relatively non-elastic material. In particular aspects, the inner layer 102 and/or the outer layer 108 can comprise a polymeric material having a modulus of elasticity of 400 MPa or greater. Exemplary materials can include ultra-high-molecular-weight polyethylene (UHMWPE) (e.g., Dyneema®), high-molecular-weight polyethylene (HMWPE), or polyether ether ketone (PEEK). With regard to the inner layer 102 in particular, such low coefficient of friction materials can facilitate passage of the prosthetic device through the lumen 112. Other suitable materials for the inner and outer layers can include polyimide, polytetrafluoroethylene (PTFE), expanded polytetrafluoroethylene (ePTFE), ethylene tetrafluoroethylene (ETFE), nylon, polyethylene, polyamide, polyether block amide (e.g., Pebax), and/or combinations of any of the above. Some aspects the sheath 8 can include a lubricious liner on the inner surface of the inner layer 102. Examples of suitable lubricious liners include materials that can further reduce the coefficient of friction of the inner layer 102, such as PTFE, polyethylene, polyvinylidine fluoride, and combinations thereof. Suitable materials for a lubricious liner also include other materials desirably having a coefficient of friction of 0.1 or less.

Additionally, some aspects of the sheath 8 can include an exterior hydrophilic coating on the outer surface of the outer layer 108. Such a hydrophilic coating can facilitate insertion of the sheath 8 into a patient's vessel, reducing potential damage. Examples of suitable hydrophilic coatings include the Harmony™ Advanced Lubricity Coatings and other Advanced Hydrophilic Coatings available from SurModics, Inc., Eden Prairie, MN. DSM medical coatings (available from Koninklijke DSM N.V, Heerlen, the Netherlands), as well as other hydrophilic coatings (e.g., PTFE, polyethylene, polyvinylidene fluoride), are also suitable for use with the sheath 8. Such hydrophilic coatings may also be included on the inner surface of the inner layer 102 to reduce friction between the sheath and the delivery system, thereby facilitating use and improving safety. In some aspects, a hydrophobic coating, such as Perylene, may be used on the outer surface of the outer layer 108 or the inner surface of the inner layer 102 in order to reduce friction.

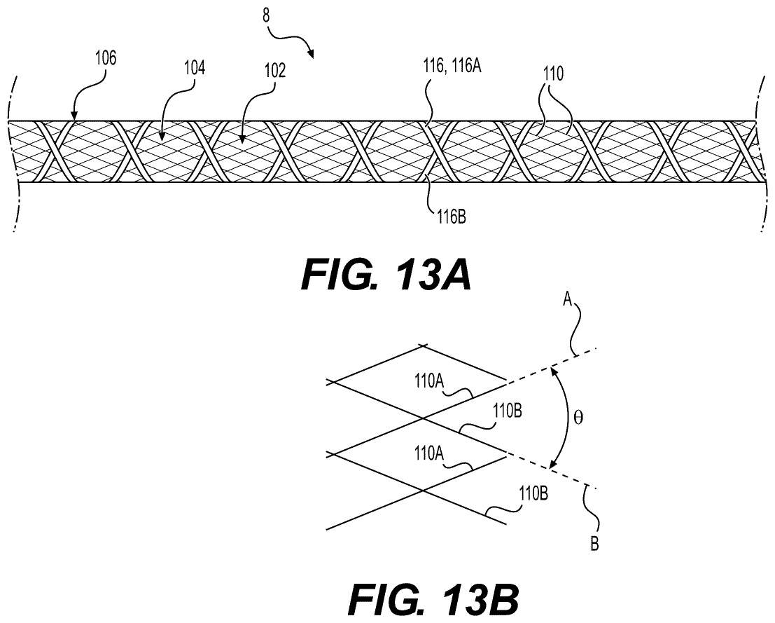

In certain aspects, the second layer 104 can be a braided layer. FIGS. 13A and 13B illustrate the sheath 8 with the outer layer 108 removed to expose the elastic third layer 106. With reference to FIGS. 13A and 13B, the braided second layer 104 can comprise a plurality of members or filaments 110 (e.g., metallic or synthetic wires or fibers) braided together. The braided second layer 104 can have any desired number of filaments 110, which can be oriented and braided together along any suitable number of axes. For example, with reference to FIG. 13B, the filaments 110 can include a first set of filaments 110A oriented parallel to a first axis A, and a second set of filaments 110B oriented parallel to a second axis B. The filaments 110A and 110B can be braided together in a biaxial braid such that the first set of filaments 110A oriented along axis A form an angle θ with the second set of filaments 110B oriented along axis B. In certain aspects, the angle θ can be from 5° to 70°, 10° to 60°, 10° to 50°, or 10° to 45°. In the illustrated example, the angle θ is 45°. In other aspects, the filaments 110 can also be oriented along three axes and braided in a triaxial braid, or oriented along any number of axes and braided in any suitable braid pattern. The braided second layer 104 can extend along substantially the entire length L of the sheath 8, or alternatively, can extend only along a portion of the length of the sheath. In particular aspects, the filaments 110 can be wires made from metal (e.g., Nitinol, stainless steel, etc.), or any of various polymers or polymer composite materials, such as carbon fiber. In certain aspects, the filaments 110 can be round, and can have a diameter of from 0.01 mm to 0.5 mm, 0.03 mm to 0.4 mm, or 0.05 mm to 0.25 mm. In other aspects, the filaments 110 can have a flat cross-section with dimensions of 0.01 mm×0.01 mm to 0.5 mm×0.5 mm, or 0.05 mm×0.05 mm to 0.25 mm×0.25 mm. In one aspect, filaments 110 having a flat cross-section can have dimensions of 0.1 mm×0.2 mm. However, other geometries and sizes are also suitable for certain aspects. If braided wire is used, the braid density can be varied. Some aspects have a braid density of from ten picks per inch to eighty picks per inch, and can include eight wires, sixteen wires, or up to fifty-two wires in various braid patterns. In other aspects, the second layer 104 can be laser cut from a tube, or laser-cut, stamped, punched, etc., from sheet stock and rolled into a tubular configuration. The second layer 104 can also be woven or knitted, as desired.

The third layer 106 can be a resilient, elastic layer (also referred to as an elastic material layer). In certain aspects, the elastic third layer 106 can be configured to apply radially inward force to the underlying inner layer 102 and second 104 in a radial direction (e.g., toward the central axis 114 of the sheath) when the sheath expands beyond its natural diameter by passage of the delivery apparatus through the sheath. Stated differently, the elastic third layer 106 can be configured to apply encircling/radially inward pressure to the layers of the sheath beneath the elastic third layer 106 to counteract expansion of the sheath. The radially inwardly directed force is sufficient to cause the sheath to collapse radially back to its unexpanded state after the delivery apparatus is passed through the sheath.

In the illustrated example, the elastic third layer 106 can comprise one or more members configured as strands, ribbons, or bands 116 helically wrapped around the braided second layer 104. For example, in the illustrated aspect the elastic third layer 106 comprises two elastic bands 116A and 116B wrapped around the braided second layer 104 with opposite helicity, although the elastic layer may comprise any number of bands depending upon the desired characteristics. The elastic bands 116A and 116B can be made from, for example, any of a variety of natural or synthetic elastomers, including silicone rubber, natural rubber, any of various thermoplastic elastomers, polyurethanes such as polyurethane siloxane copolymers, urethane, plasticized polyvinyl chloride (PVC), styrenic block copolymers, polyolefin elastomers, etc. In some aspects, the elastic layer can comprise an elastomeric material having a modulus of elasticity of 200 MPa or less. In some aspects, the elastic third layer 106 can comprise a material exhibiting an elongation to break of 200% or greater, or an elongation to break of 400% or greater. The elastic third layer 106 can also take other forms, such as a tubular layer comprising an elastomeric material, a mesh, a shrinkable polymer layer such as a heat-shrink tubing layer, etc. In lieu of, or in addition to, the elastic third layer 106, the sheath 8 may also include an elastomeric or heat-shrink tubing layer around the outer layer 108. Examples of such elastomeric layers are disclosed in U.S. Publication No. 2014/0379067, U.S. Publication No. 2016/0296730, and U.S. Publication No. 2018/0008407, which are incorporated herein by reference. In other aspects, the elastic third layer 106 can also be radially outward of the polymeric outer layer 108.

In certain aspects, one or both of the inner layer 102 and/or the outer layer 108 can be configured to resist axial elongation of the sheath 8 when the sheath expands. More particularly, one or both of the inner layer 102 and/or the outer layer 108 can resist stretching against longitudinal forces caused by friction between a prosthetic device and the inner surface of the sheath 8 such that the length L remains substantially constant as the sheath expands and contracts. As used herein with reference to the length L of the sheath, the term “substantially constant” means that the length L of the sheath increases by not more than 1%, by not more than 5%, by not more than 10%, by not more than 15%, or by not more than 20%. Meanwhile, with reference to FIG. 13B, the filaments 110A and 110B of the braided second layer 104 can be allowed to move angularly relative to each other such that the angle θ changes as the sheath expands and contracts. This, in combination with the longitudinal folds/ridges 126 in the inner layer 102 and outer layer 108, can allow the lumen 112 of the sheath to expand as a prosthetic device is advanced through it.

For example, in some aspects the inner layer 102 and the outer layer 108 can be heat-bonded during the manufacturing process such that the braided second layer 104 and the elastic third layer 106 are encapsulated between the inner layer 102 and outer layer 108. More specifically, in certain aspects the inner layer 102 and the outer layer 108 can be adhered to each other through the spaces between the filaments 110 of the braided second layer 104 and/or the spaces between the elastic bands 116. The inner layer 102 and outer layer 108 can also be bonded or adhered together at the proximal and/or distal ends of the sheath. In certain aspects, the inner layer 102 and outer layer 108 are not adhered to the filaments 110. This can allow the filaments 110 to move angularly relative to each other, and relative to the inner layer 102 and outer layer 108, allowing the diameter of the braided second layer 104, and thereby the diameter of the sheath, to increase or decrease. As the angle θ between the filaments 110A and 110B changes, the length of the braided second layer 104 can also change. For example, as the angle θ increases, the braided second layer 104 can foreshorten, and as the angle θ decreases, the braided second layer 104 can lengthen to the extent permitted by the areas where the inner layer 102 and outer layer 108 are bonded. However, because the braided second layer 104 is not adhered to the inner layer 102 and outer layer 108, the change in length of the braided layer that accompanies a change in the angle θ between the filaments 110A and 110B does not result in a significant change in the length L of the sheath.

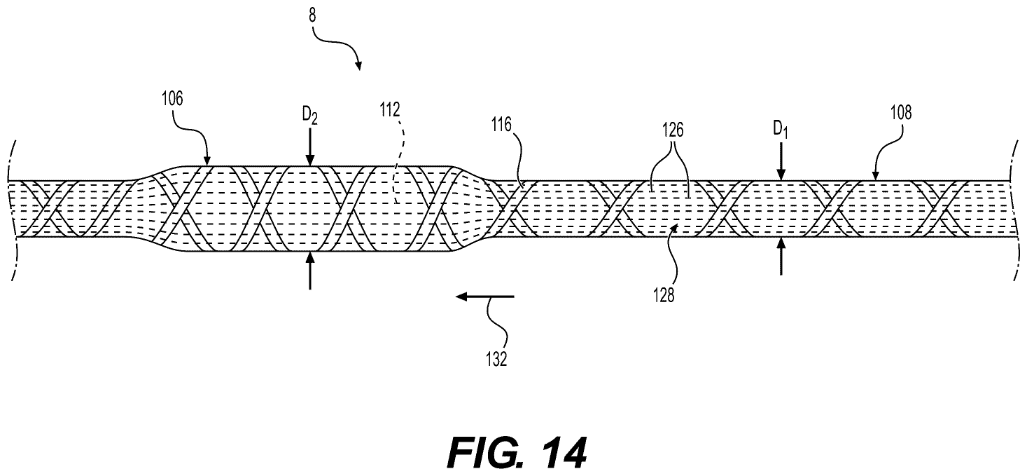

FIG. 14 illustrates radial expansion of the sheath 8 as a prosthetic device (e.g., implant 12) is passed through the sheath 8 in the direction of arrow 132 (e.g., distally). As the prosthetic device (implant 12) is advanced through the sheath 8, the sheath can resiliently expand to a second diameter D₂ that corresponds to a size or diameter of the prosthetic device. As the prosthetic device (implant 12) is advanced through the sheath 8, the prosthetic device can apply longitudinal force to the sheath in the direction of motion by virtue of the frictional contact between the prosthetic device and the inner surface of the sheath. However, as noted above, the inner layer 102 and/or the outer layer 108 can resist axial elongation such that the length L of the sheath remains constant, or substantially constant. This can reduce or prevent the braided layer second 104 from lengthening, and thereby constricting the lumen 112.

Meanwhile, the angle θ between the filaments 110A and 110B can increase as the sheath expands to the second diameter D₂ to accommodate the prosthetic valve. This can cause the braided second layer 104 to foreshorten. However, because the filaments 110 are not engaged or adhered to the inner layer 102 or outer layer 108, the shortening of the braided second layer 104 attendant to an increase in the angle θ does not affect the overall length L of the sheath. Moreover, because of the longitudinally-extending folds/ridges 126 formed in the layers 102 and 108, the inner layer 102 and outer layer 108 can expand to the second diameter D₂ without rupturing, in spite of being relatively thin and relatively non-elastic. In this manner, the sheath 8 can resiliently expand from its natural diameter D₁ to a second diameter D₂ that is larger than the diameter D₁ as a prosthetic device is advanced through the sheath, without lengthening, and without constricting. Thus, the force required to push the prosthetic implant through the sheath is significantly reduced.

Additionally, because of the radial force applied by the elastic third layer 106, the radial expansion of the sheath 8 can be localized to the specific portion of the sheath occupied by the prosthetic device. For example, with reference to FIG. 14, as the prosthetic device (implant 12) moves distally through the sheath 8, the portion of the sheath immediately proximal to the prosthetic device (e.g., implant 12) can radially collapse back to the initial diameter D₁ under the influence of the elastic third layer 106. The inner layer 102 and outer layer 108 can also buckle as the circumference of the sheath is reduced, causing the ridges 126 and the valleys 128 to reform. This can reduce the size of the sheath required to introduce a prosthetic device of a given size. Additionally, the temporary, localized nature of the expansion can reduce trauma to the blood vessel into which the sheath is inserted, along with the surrounding tissue, because only the portion of the sheath occupied by the prosthetic device expands beyond the sheath's natural diameter and the sheath collapses back to the initial diameter once the device has passed. This limits the amount of tissue that must be stretched in order to introduce the prosthetic device, and the amount of time for which a given portion of the vessel must be dilated.

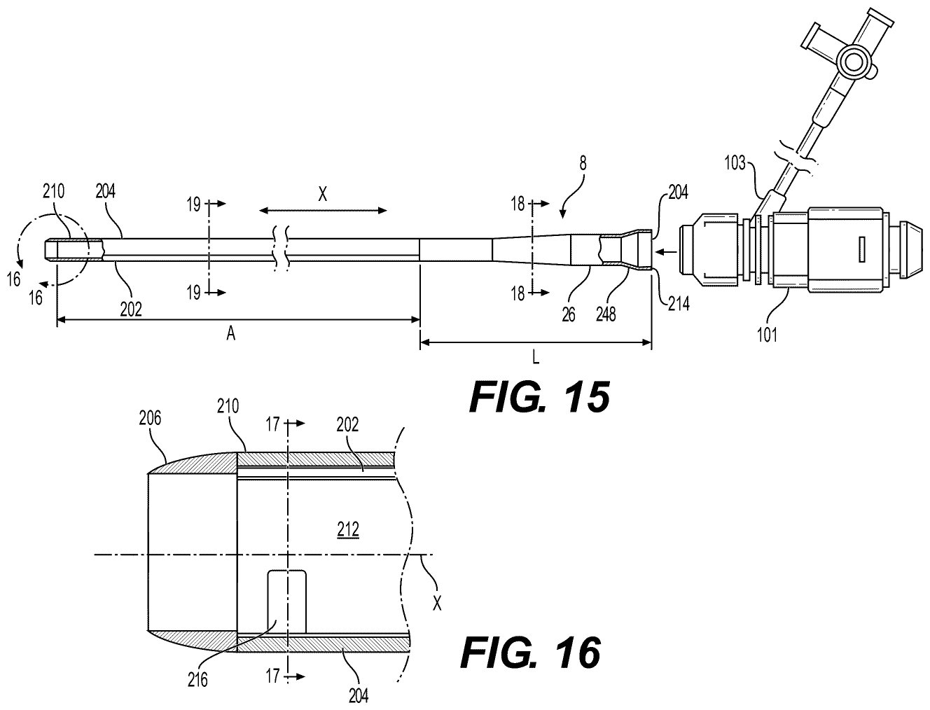

In another example layered sheath 8 structure, FIGS. 15-23 illustrate various features of the coaxial layered structure of the expandable sheath 8 of FIG. 1 according to another aspect. Similar reference numbers are used to describe like elements. It is to be understood that the variations (e.g., materials and alternate configurations) described above with reference to FIGS. 11-14 can also apply to the example shown in FIGS. 15-23. Furthermore, the variations described below with reference to FIGS. 15-23 can also be applied to the sheath described in FIGS. 11-14.

Similar to various aspects of the sheath 8 described above in reference to FIGS. 11-14, the sheath 8 of FIGS. 15-23 includes a plurality of layers. For example, the sheath 8 illustrated in FIGS. 15-23, also includes an inner layer 202 and an outer layer 204 disposed around the inner layer 202. The inner layer 202 can define a lumen 212 through which the delivery apparatus travels into the patient's vessel in order to deliver, remove, repair, and/or replace a prosthetic device, moving in a direction along the longitudinal axis X. Similar to the sheath illustrated in FIGS. 11-14, as the prosthetic device passes through the sheath 8, the sheath 8 locally expands from a first, resting/unexpanded diameter to a second, expanded diameter to accommodate the prosthetic device. After the prosthetic device passes through a particular location of the sheath 8, each successive expanded portion or segment of the sheath 8 at least partially returns to the smaller, resting/unexpanded diameter. In this manner, the sheath 8 can be considered self-expanding, in that it does not require use of a balloon, dilator, and/or obturator to expand.

Similar to the examples above, the inner and outer layers 202, 204 can comprise any suitable materials. Suitable materials for the inner layer 202 include polytetrafluoroethylene (PTFE), ethylene tetrafluoroethylene (ETFE), nylon, polyethylene, polyether block amide (e.g., Pebax), and/or combinations thereof. In one specific implementation the inner layer 202 can comprise a lubricious, low-friction, or hydrophilic material, such as PTFE. Such low coefficient of friction materials can facilitate passage of the prosthetic device through the lumen defined by the inner layer 202. In some examples, the inner layer 202 can have a coefficient of friction of less than about 0.1. Some examples of the sheath 8 can include a lubricious liner on the inner surface of the inner layer 202. Examples of suitable lubricious liners include materials that can further reduce the coefficient of friction of the inner layer 202, such as PTFE, polyethylene, polyvinylidene fluoride, and combinations thereof. Suitable materials for a lubricious liner also include other materials desirably having a coefficient of friction of about 0.1 or less.

Suitable materials for the outer layer 204 include nylon, polyethylene, Pebax, HDPE, polyurethanes (e.g., Tecoflex), and other medical grade materials. In one implementation, the outer layer 204 can comprise high density polyethylene (HDPE) and Tecoflex (or other polyurethane material) extruded as a composite. In some implementations, the Tecoflex can act as an adhesive between the inner layer 202 and the outer layer 204 and may only be present along a portion of the inner surface of the outer layer 204. Other suitable materials for the inner and outer layers are also disclosed in U.S. Pat. Nos. 8,690,936 and 8,790,387, which are incorporated herein by reference.

Additionally, some examples of the sheath 8 include an exterior hydrophilic coating on the outer surface of the outer layer 204. Such a hydrophilic coating can facilitate insertion of the sheath 100 into a patient's vessel. Examples of suitable hydrophilic coatings include the Harmony™ Advanced Lubricity Coatings and other Advanced Hydrophilic Coatings available from SurModics, Inc., Eden Prairie, MN. DSM medical coatings (available from Koninklijke DSM N. V, Heerlen, the Netherlands), as well as other hydrophilic coatings (e.g., PTFE, polyethylene, polyvinylidene fluoride), are also suitable for use with the sheath 100.

FIG. 16 provides a partial cross-section of the distal end of the sheath 8 along section line 16-16 identified in FIG. 15. As described herein, the sheath 8 can be inserted into a vessel (e.g., the femoral or iliac arteries) by passing through the skin of patient, such that a soft tip portion 206 at the distal end 210 of the sheath 8 is inserted into the vessel. As best seen in FIG. 16, the soft tip portion 206 can comprise, in some examples, low density polyethylene (LDPE) and can be configured to minimize trauma or damage to the patient's vessels as the sheath is navigated through the vasculature. For example, the soft tip portion 206 can be slightly tapered to facilitate passage through the vessels. The soft tip portion 206 can be secured to the distal end 210 of the sheath 8, such as by thermally bonding the soft tip portion 206 to the inner and outer layers of the sheath 8. Such a soft tip portion 206 can be provided with a lower hardness than the other portions of the sheath 8. In some examples, the soft tip potion 206 can have a Shore hardness from about 25 D to about 40 D. The soft tip portion 206 is configured to be radially expandable to allow a prosthetic device to pass through the distal opening of the sheath 8. For example, the soft tip portion 206 can be formed with a weakened portion, such as an axially extending score line or perforated line that is configured to split and allow the soft tip portion 206 to expand radially when the prosthetic device passes therethrough.

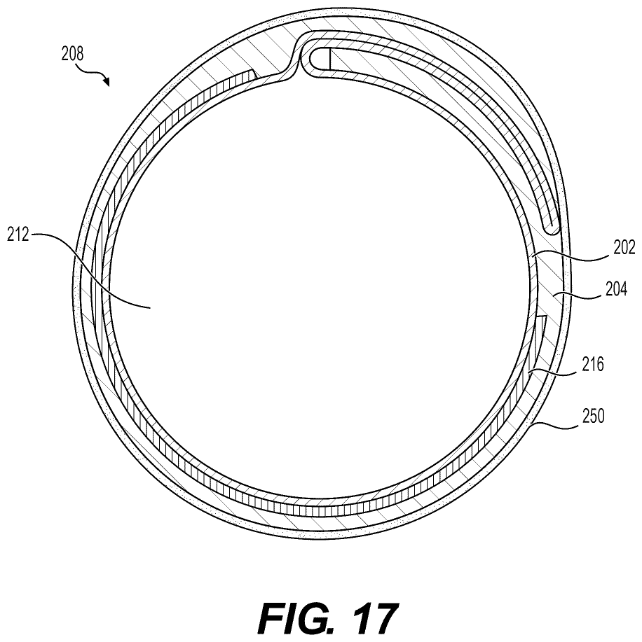

FIG. 17 shows a cross-section view of the sheath 8 taken near the distal end 210 of the sheath 8 as indicated by section line 17-17 in FIG. 16. As illustrated in FIGS. 16 and 17, the sheath 8 can include at least one radiopaque filler or marker, such as a discontinuous, or C-shaped, band/marker 216 positioned near the distal end 210 of the sheath 8. The marker 216 can be associated with the inner and/or outer layers 202, 204 of the sheath 8. For example, as shown in FIG. 17, the marker 216 can be positioned between the inner layer 202 and the outer layer 204. In alternative examples, the marker 216 can be associated with the outer surface of the outer layer 204. In some examples, the marker 216 can be embedded or blended within the inner or outer layers 202, 204.

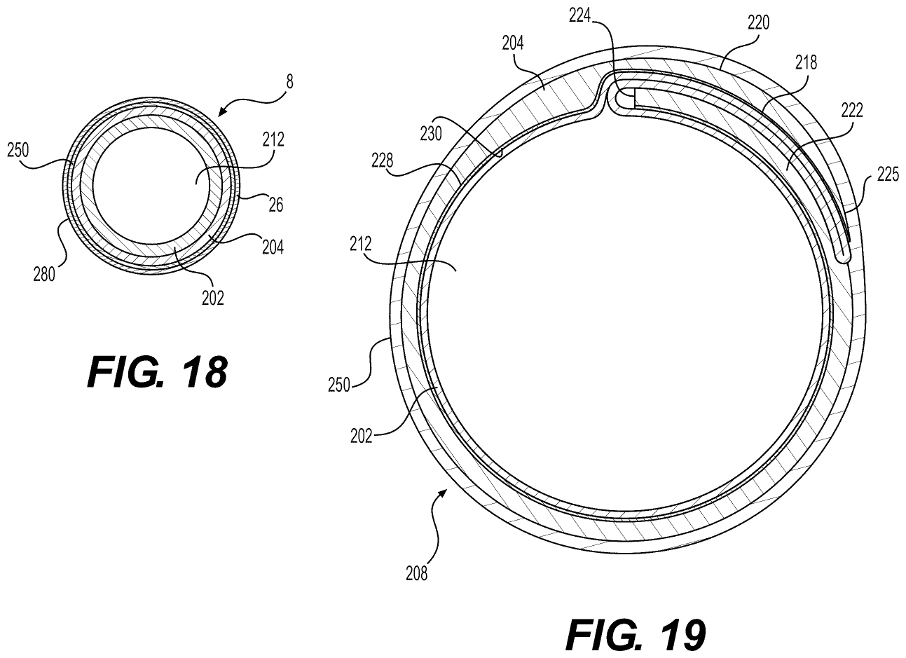

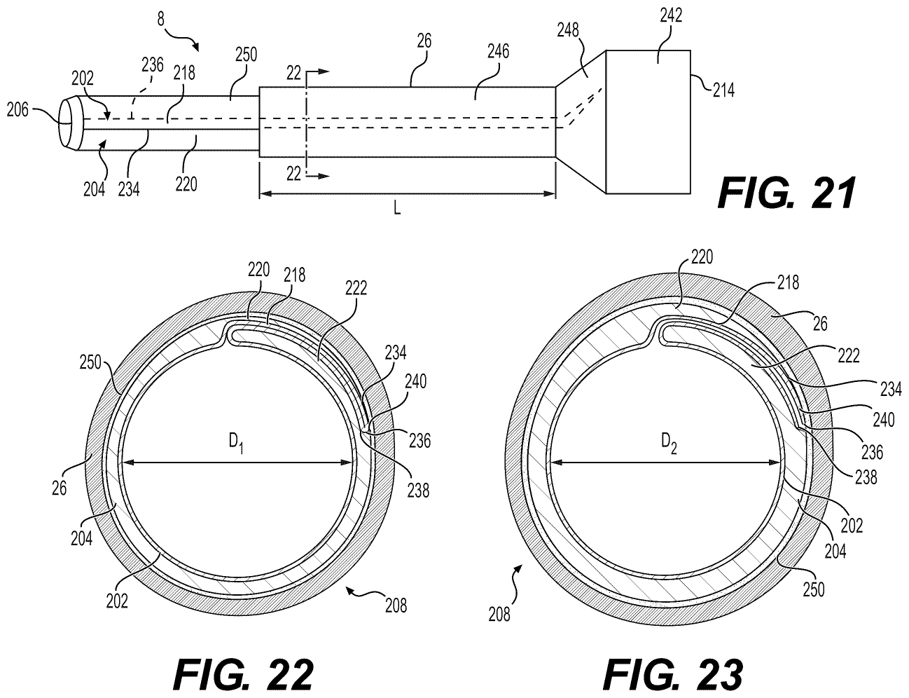

FIGS. 18 and 19 show additional cross sections taken at different points along the sheath 8. FIG. 18 shows a cross-section of a segment of the sheath near the proximal end 214 of the sheath 8, as indicated by section line 18-18 in FIG. 15. At this location, the sheath 8 includes the inner layer 202, outer layer 204, elastic outer layer 250/outer jacket, and the strain relief layer 26. At this location, near the proximal end of the sheath 8, the inner and outer layers 202, 204 are substantially tubular. Here the inner and outer layers 202, 204 can be formed without any slits or folded portions in the layers. By contrast, as described below, the inner and outer layers 202, 204 at different locations along the sheath 8 (e.g., at the point indicated by section line 19-19 in FIG. 15 and/or the point indicated by section line 22-22 in FIG. 21) can have a different configuration.

As shown in FIG. 19, the inner layer 202 can be arranged to form a substantially cylindrical lumen 212 therethrough. Inner layer 202 can include one or more folded portions 218. In the implementation shown in FIG. 19, inner layer 202 is arranged to have one folded portion 218 that can be positioned on either side of the inner layer 202. Inner layer 202 can be continuous, in that there are no breaks, slits, or perforations in inner layer 202. Outer layer 204 can be arranged in an overlapping fashion such that an overlapping portion 220 overlaps at least a part of the folded portion 218 of the inner layer 202. As shown in FIG. 19, the overlapping portion 220 also overlaps an underlying portion 222 of the outer layer 204. The underlying portion 222 can be positioned to underlie both the overlapping portion 220 of the outer layer 204, as well as the folded portion 218 of the inner layer 202. Thus, the outer layer 204 can be discontinuous, in that it includes a slit or a cut in order to form the overlapping and underlying portions 220, 222. In other words, a first edge 224 of the outer layer 204 is spaced apart from a second edge 225 of the outer layer 204 so as not to form a continuous layer.

As shown in FIG. 19, the sheath 8 can also include a thin layer of bonding or adhesive material 228 positioned between the inner and outer layers 202, 204. In one implementation, the adhesive material 228 can comprise a polyurethane material such as Tecoflex. The adhesive material 228 can be positioned on an inner surface 202 of at least a portion of the outer layer 204 so as to provide adhesion between selected portions of the inner and outer layers 202, 204. For example, the outer layer 204 may only include a Tecoflex layer/adhesive material 228 around the portion of the inner surface 230 that faces the lumen-forming portion of the inner layer 202. In other words, the Tecoflex layer/adhesive material 228 can be positioned so that it does not contact the folded portion 218 of the inner layer 202 in some implementations. In other implementations, the Tecoflex layer/adhesive material 228 can be positioned in different configurations as desired for the particular application. For example, as shown in FIG. 19, the Tecoflex layer/adhesive material 228 can be positioned along the entire inner surface 230 of the outer layer 204. In an alternative example, the Tecoflex layer can be applied to the outer surface of the inner layer 202 instead of the inner surface of the outer layer 204. The Tecoflex layer can be applied to all or selected portions on the inner layer 202; for example, the Tecoflex layer can be formed only on the portion of the inner layer 202 that faces the lumen-forming portion of the outer layer 204 and not on the folded portion 218. The configuration of FIG. 19 allows for radial expansion of the sheath 8 as an outwardly directed radial force is applied from within (e.g., by passing a medical device such as a prosthetic heart valve through the lumen 212). As radial force is applied, the folded portion 218 can at least partially separate, straighten, and/or unfold, and/or the overlapping portion 220 and the underlying portion 220 of the outer layer 204 can slide circumferentially with respect to one another, thereby allowing the diameter of lumen 212 to enlarge.

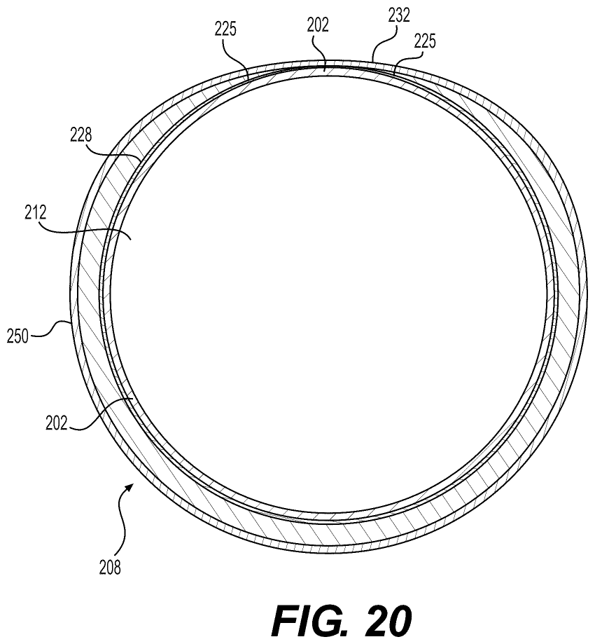

In this manner, the sheath 8 is configured to expand from a resting/unexpanded configuration (FIG. 19) to an expanded configuration shown in FIG. 20. In the expanded configuration, as shown in FIG. 20, an annular gap 232 can form between the longitudinal edges of the overlapping portion 220 and the underlying portion 222 of the outer layer 204. As the sheath 8 expands at a particular location, the overlapping portion 220 of the outer layer 204 can move circumferentially with respect to the underlying portion 222 as the folded portion 218 of the inner layer 202 unfolds. This movement can be facilitated by the use of a low-friction material for inner layer 202, such as PTFE. Further, the folded portion 218 can at least partially separate and/or unfold to accommodate a medical device having a diameter larger than that of lumen 212 in the resting/unexpanded configuration. As shown in FIG. 20, in some implementations, the folded portion of the inner layer 102 can completely unfold, so that the inner layer 102 forms a cylindrical tube at the location of the expanded configuration.

Similar to the example sheath 8 in FIG. 14, the sheath 8 is configured to locally expands at a particular location corresponding to the location of the medical device along the length of the lumen 212, and then locally contracts once the medical device has passed that particular location. Thus, a bulge may be visible, traveling longitudinally along the length of the sheath 8 as a medical device is introduced through the sheath 8, representing continuous local expansion and contraction as the device travels the length of the sheath 8. Each segment of the sheath 8 will locally contract after removal of any radial outward force such that the sheath 8 at least partially returns to the original resting/unexpanded diameter of lumen 212. Similar to the example sheath described above, an elastic outer layer 250 can (optionally) be provided along the sheath 8, urging the inner and outer layers 202, 204 back towards the unexpanded configuration.

The layers 202, 204 of sheath 8 can be configured having the folded portion 218 as shown in FIG. 19 along at least a portion of the length of the sheath 8. In some examples, the inner and outer layers 202, 204 can be configured as shown in FIG. 19 along the length A (FIG. 15) such that the folded portion 218 extends from a location adjacent the soft tip portion 206 to a location closer to the proximal end 214 of the sheath 8, adjacent and/or under the distal end of the strain relief layer 26. In this matter, the sheath 8 is expandable and contractable only along a portion of the length of the sheath corresponding to length A (which typically corresponds to the section of the sheath inserted into the narrowest section of the patient's vasculature).

In some examples, the folded portion 218 portion extends from a location adjacent the soft tip portion 206 under the strain relief layer 26, as illustrated in FIG. 21. In this example, the folded structure of the inner layer 202 extends from the soft tip portion 206, under the strain relief layer 26 and along the tapered segment 248 of the strain relief layer 26.

FIGS. 22 and 23 illustrate cross-section views of the sheath 8 taken along the strain relief layer 26 at section line 22-22 in FIG. 21. In this example, the folded portion 218 of the inner layer 202 extends under the strain relief layer 26. FIG. 22 shows a cross-section of the sheath 8 in a resting/unexpanded configuration having an inner diameter D₁. FIG. 23 shows a cross-section of the sheath 8 in a (partially) expanded configuration, having an inner diameter D₂, where D₂ is greater than D₁.

As shown in FIGS. 22-23, in some examples, the overlapping portion 220 does not overlap the entire folded portion 218 of the inner layer 202, and thus a portion of the folded portion 218 can be directly adjacent to the strain relief layer 26 in locations where the strain relief layer 26 is present. In locations where the strain relief layer 26 is not present, part of the folded portion 218 may be visible from the outside of the sheath 8, as seen in FIG. 21 (and/or visible through an elastic outer layer 250 described in more detail below). In these examples, the sheath 8 can include a longitudinal seam 234 where the overlapping portion 220 terminates at the folded portion 218. In use, the sheath 8 can be positioned such that the seam 234 is posterior to the point of the sheath that is 180 degrees from the seam 234 (e.g., facing downward in the view of FIG. 21). As shown in FIG. 21, the seam 234 need not extend the entire length of the sheath 8, and end at a transition point between portions of the sheath having a folded inner layer and portions of the sheath not having a folded inner layer.

In some examples, the folded portion 218 can include a weakened portion 236, such as a longitudinal perforation, score line, and/or slit, along at least a portion of the length of the inner layer 202. The weakened portion 236/slit allows for the two adjacent ends 238, 240 of the folded portion 218/inner layer 202 to move relative to one another as the sheath 8 expands to the expanded configuration shown in FIG. 23. For example, the sheath 8 locally expands as a medical device is inserted therethrough, causing the weakened portion 236 to split/separate.

As illustrated in FIGS. 2, 15 and 21, the sheath 8 includes a strain relief layer 26. The strain relief layer 26/tube is provided adjacent the proximal end of the sheath 8 extends along/over the outer surface of the sheath 8. In some examples, the strain relief layer 26 is provided over the outer layer 108, 204 of the sheath 8. The strain relief layer 26 forms a smooth transition between the sheath hub 20 and the sheath 8 and facilitates mating of the sheath 8 with the sheath hub 20.

Additionally, and as will be described in more detail below, the strain relief layer 26 provides a region of higher durometer or stiffness that restricts expansion of the underlying sheath layers. This helps to ensure hemostasis between the portions of the sheath 8 inside the patient and the sheath hub (external to the patient). The increased durometer and/or stiffness along the strain relief layer 26 prevents blood from flowing between the various layers of the sheath 8 exterior to the patient during the procedure, helping to withstand the blood pressure that would otherwise cause the sheath to “balloon up” with body fluid/blood. Additionally, the strain relief layer 26 can be sized and configured to form a seal with the patient's artery when inserted, such that blood is substantially prevented from flowing between the strain relief layer 26 and the vessel wall. For example, although the strain relief layer 26 does not extend all the way to the distal end 210 of the sheath 8, the strain relief layer 26 can extend distally enough along the sheath 8 that when the sheath 8 is fully inserted into the patient a portion of the strain relief layer 26 extends through and seals against the arteriotomy site.

As described above, the strain relief layer 26 is provided over the outer layer 108, 204 of the sheath 8. The strain relief layer 26 can be bonded to the outer layer 108, 204 to prevent the strain relief layer 26 from sliding over the outer layer and “bunching up” in response to the friction forces applied by the surrounding tissue during insertion of the sheath 8 into the patient's vasculature. For example, the strain relief layer 26 can be bonded at the proximal end and/or distal end of the outer layer 108, 204. At the proximal and distal ends, the strain relief layer 26 can be bonded to the outer layer 204 around the full circumference of the outer layer. At the distal end of the sheath 8, the strain relief layer 26 can alternatively be bonded to the inner layer(s) of the sheath 8. For example, the strain relief layer 26 can be bonded to the distal end surface of the inner layer 102, 202.

FIGS. 18, 22 and 23 illustrate cross-section views of the sheath 8 along the strain relief layer 26. FIG. 18 shows a cross-section of a segment of the sheath near the proximal end 214 of the sheath 8, as indicated by section line 18-18 in FIG. 15. Similarly, FIGS. 22 and 23 show cross-section segments of various example sheaths near the proximal end 214 of the sheath 8 and closer to the distal end of the strain relief layer 26, as indicated by section line 22-22 in FIG. 21. As illustrated in each of FIGS. 15-23, the sheath 8 at this location can comprise an inner layer (liner) 202, outer layer 208, adhesive layer/adhesive material 228, an optional elastic outer layer 250, and the strain relief layer 26.

The strain relief layer 26 extends circumferentially around at least a portion of the inner layer 202 and outer layer 204. The strain relief layer 26 extends from the proximal end 214 of the sheath 8 towards the distal end 210 of the sheath 8. As shown in FIG. 21 (and FIG. 15), the strain relief layer 26 extends for a length L along at least a portion of the main body of the sheath 8. In further examples, the strain relief layer 26 extends to a point adjacent the distal end 210, or can extend all the way to the distal end 210 of sheath 8. In some examples, the longitudinal length L of the strain relief layer 26 can range from about 10 cm to the entire length of the sheath 8.

The strain relief layer 26 extends to/adjacent the proximal end 214 of the sheath 8 and provides a compression fit over the distal end of the sheath hub 20 thereby coupling the sheath 8 to the sheath hub 20. Additionally or alternatively, the strain relief layer 26 secured between the sheath hub 20 and the sheath hub cap 22 or other fastening device for by coupling the proximal end of the sheath to the sheath hub 20. In some examples, the strain relief layer 26 does not extend all the way to the proximal end 214 of the sheath 8.

It is understood that strain relief layer 26, as shown herein, can have similar composition and characteristics of the inner and outer layers as disclosed herein. Various compositions are disclosed, for example, in Application No. PCT/US2021/301275, entitled “Expandable sheath for introducing an endovascular delivery device into a body,” the disclosure of which is herein incorporated by reference.

The strain relief layer 26 can comprise any lubricious, low-friction, and/or relatively non-elastic material. Preferably the materials used can include high durometer polymers, with low elasticity. In some examples, the strain relief layer 26 is composed of the same and/or similar material to the inner layer 202 and/or outer layer 204. For example, as described above regarding the inner and/or outer layer 102, 108, exemplary materials can include polyurethane (e.g., high density polyethylene), ultra-high-molecular-weight polyethylene (UHMWPE) (e.g., Dyneema®), high-molecular-weight polyethylene (HMWPE), or polyether ether ketone (PEEK). Other suitable materials for strain relief layer 26 can include polyimide, polytetrafluoroethylene (PTFE), expanded polytetrafluoroethylene (ePTFE), ethylene tetrafluoroethylene (ETFE), nylon, polyethylene, polyamide, polyether block amide (e.g., Pebax), and/or combinations of any of the above. Materials for the strain relief layer 26 can be selected such that it impedes expansion of the underlying layers of the sheath 8.

The strain relief layer 26 can have a thickness ranging from, for example, about 0.001″ to about 0.010.″ In some implementations, the strain relief layer 26 can have a thickness of from about 0.003″ to about 0.006.″ The wall thickness is measured radially between the inner surface of the strain relief layer 26 and the outer surface of the strain relief layer 26.

In alternative examples, the material composition and/or wall thickness can change along the length of the strain relief layer 26. For example, the strain relief layer 26 can be provided with one or more segments, where the composition and/or thickness changes from segment to segment. In an example aspect, the Durometer rating of the composition changes along the length of the strain relief layer 26 such that segments near the proximal end comprise a stiffer material or combination of materials, while segments near the distal end comprise a softer material or combination of materials. Similarly, the wall thickness of the strain relief layer 26 in segments near the proximal end can be thicker/greater than the wall thickness of the outer layer 250 near the distal end.

As illustrated in FIGS. 15 and 21, the strain relief layer 26 has a proximal end and a distal end and a central lumen extending longitudinally therethrough. The strain relief layer 26 includes a generally tubular shaped proximal portion 242 adjacent the proximal end of the strain relief layer 26, and a generally tubular shaped distal portion 246 adjacent the distal end of the strain relief layer 26. The strain relief layer 26 includes a frustoconical shaped tapered segment 248 extending between the proximal portion 242 and the distal portion 246 of the strain relief layer 26, such that the diameter of the strain relief layer 26 at the proximal portion 242 is greater than the diameter of the strain relief layer 26 at the distal portion 246 of the strain relief layer 26. The tapered segment 248 and the flared proximal portion 242 help ease the transition of the medical device/delivery system when passing between the larger diameter sheath hub 20 to the smaller diameter of the sheath 8.

As described above, the strain relief layer 26 is made of a material that is stiffer than the other sheath 8 layers such that the strain relief layer 26 inhibits expansion of the portion of the sheath disposed along/under the strain relief layer 26. Because radial expansion is limited along the strain relief layer 26, higher push forces are necessary to advance the medical device (implant 12) through the central lumen of the sheath 8. In some examples, the highest push force through the sheath 8 is experienced near the ends (e.g., proximal and distal ends) of the strain relief layer 26. The thickness and/or composition of the strain relief layer 26 can be adjusted to improve the performance of the strain relief layer 26 and to reduce the push force.

The elastic outer layer 250 can be positioned around at least a portion of the strain relief layer 26, outer layer 108, 204 and/or the inner layers of the sheath 8. As illustrated in FIGS. 21-23, the outer layer 250 can surround the entire circumference of outer layer 204, and can extend longitudinally along any portion of the length of the sheath 8, including along (over or under) the strain relief layer 26. The elastic outer layer 250 extends for a length along at least a portion of the main body of the sheath 8. In some examples, the elastic outer layer 250 extends to a point adjacent the distal end 210, or can extend all the way to the distal end 210 of sheath 8. For example, the elastic outer layer 250 extends over the entire length of the sheath 8.

As shown in FIGS. 17-20, 22 and 23, the elastic outer layer 250 can be a continuous tubular layer, without slits or other discontinuities. The elastic outer layer 250 extends between strain relief layer 26 and the outer surface of the outer layer 204. In other examples, the elastic outer layer 250 extends over the outer surface of the strain relief layer 26 and the outer surface outer layer 204. In further examples, the elastic outer layer 250 extends both over the strain relief layer 26 and/or between the outer layer of the sheath 8 and the strain relief layer 26.

The elastic outer layer 250 can comprise any pliable, elastic material(s) that expand and contract, preferably with a high expansion ratio. Preferably, the materials used can include low durometer polymers with high elasticity, such as Pebax, polyurethane, silicone, and/or polyisoprene. Materials for the elastic outer layer 250 can be selected such that it does not impede expansion of the inner and outer layers of the sheath 8. The elastic outer layer 250 can have a thickness ranging from, for example, about 0.001″ to about 0.010.″ In some implementations, the elastic outer layer 250 can have a thickness of from about 0.003″ to about 0.006.″ The elastic outer layer 250 can be configured to stretch and expand as the sheath expands, as shown in the expanded configuration in FIG. 20.

A method of delivering a medical device through a sheath (e.g., to a procedure site) is described below. When used to deliver a medical device to a treatment site within a patient, the sheath 8 is inserted at least partially into the blood vessel of the patient and the distal end of the sheath 8 is positioned at a location proximate the treatment site. A tapered introducer 6 can be positioned within sheath 8 to facilitate insertion into the treatment site. In this example, the sheath 8 is coupled to the introducer 6 such that axial movement between the introducer 6 and the sheath 8 is eliminated. Preventing movement and gapping between the introducer 6 and the sheath 8 during insertion reduces the risk of trauma to the patient's vasculature. FIG. 2 shows the example device for delivering the prosthetic device.

The method includes providing an introducer hub 30 having an elongated introducer 6 coupled to the hub body 32 of the introducer hub 30. As described above, the introducer hub 30 includes a locking channel 38 disposed in the hub body 32. The sheath locking sleeve 28 is advanced to a position adjacent a distal end of the introducer hub 30 such that a guide 31 projecting from an outer surface of the sheath locking sleeve 28 is received within the opening to the locking channel 38. Advancing the sheath locking sleeve 28 to a position adjacent the distal end of the introducer hub 30 also includes advancing the introducer 6 axially within the central lumen of the expandable sheath 8.

The introducer hub 30 is then rotated in a first direction with respect to the sheath locking sleeve 28 to move the guide 31 along the locking channel 38 into a locked position. In particular, moving the guide 31 into the locked position includes rotating the introducer hub 30 to move the guide 31 along a guide portion 40 of the locking channel 38 toward a locking portion 42. Further rotation of the introducer hub 30 directs the guide 31 into the locking portion 42 of the locking channel 38, the locking portion 42 configured to securely engage the guide 31 and fix the axial position of the introducer hub 30 with respect to the sheath locking sleeve 28. Where the locking channel 38 includes a catch 44, rotation of the introducer hub 30 in the first direction causes the guide 31 to overcome the bias force of the catch 44 and advance the guide 31 beyond the catch 44 into the locking portion 42, where the catch 44 secures the guide 31 within the locking portion 42 thereby fixing the axial location of the sheath 8 with respect to the introducer 6.

The coupled sheath 8 and introducer 6 are then inserted, at least partially, into the vasculature of the patient and the distal end of the sheath 8 is positioned at a location proximate the treatment site.

Once positioned, the introducer hub 30 is rotated in a second, opposite, direction with respect to the sheath locking sleeve 28. Rotating the introducer hub 30 in the second direction causes the guide 31 to slide along the locking channel 38, from the locking portion 42 toward the guide portion 40. In particular, rotating of the introducer hub 30 in the second direction directs the guide 31 out of the locking portion 42 of the locking channel 38 and through the guide portion 40 and releases the introducer hub 30 from the sheath locking sleeve 28. Where the locking channel 38 includes a catch 44, rotation of the introducer hub 30 in the second direction causes the guide 31 to overcome the bias force of the catch 44 and advance from the locking portion 42 to the guide portion 40 of the locking channel 38. As a result, the guide 31 slides out of the locking channel 38 into the unlocked position.

The introducer hub 30 is then disengaged from the sheath locking sleeve 28 and the introducer 6 is withdrawn from the central lumen of the sheath 8. With the central lumen of the sheath 8 clear, the prosthetic device (e.g., implant 12) is advanced through the central lumen of the sheath 8. As described above, the prosthetic device (implant 12) is delivered to the procedure site via the central lumen of the sheath 8.

A method of securing a delivery sheath to an introducer in a device for prosthetic heart valve delivery device is disclosed herein. The method comprises providing an introducer hub 30 having an elongated introducer 6 coupled thereto and including a locking channel 38 disposed in the hub body 32. The sheath locking sleeve 28 is advanced to a position adjacent a distal end of the introducer hub 30 such that a guide 31 projecting from an outer surface of the sheath locking sleeve 28 is received within an opening of the locking channel 38. Advancing the sheath locking sleeve 28 to a position adjacent the distal end of the introducer hub 30 also includes advancing the introducer 6 axially within the central lumen of the expandable sheath 8.

The introducer hub 30 is then rotated in a first direction with respect to the sheath locking sleeve 28 to move the guide 31 along the locking channel 38 into the locked position. In particular, moving the guide 31 into the locked position includes rotating the introducer hub 30 to move the guide 31 along a guide portion 40 of the locking channel 38 toward a locking portion 42. Further rotation of the introducer hub 30 directs the guide 31 into the locking portion 42 of the locking channel 38, the locking portion 42 configured to securely engage the guide 31 and fix the axial position of the introducer hub 30 with respect to the sheath locking sleeve 28. Where the locking channel 38 includes a catch 44, rotation of the introducer hub 30 in the first direction causes the guide 31 to overcome the bias force of the catch 44 and advance the guide 31 beyond the catch 44 into the locking portion 42, where the catch 44 secures the guide 31 within the locking portion 42 thereby fixing the axial location of the sheath 8 with respect to the introducer 6.

To unlock the introducer hub 30 from the sheath locking sleeve 28, the introducer hub 30 is rotated in a second, opposite, direction with respect to the sheath locking sleeve 28. Rotating the introducer hub 30 in the second direction causes the guide 31 to side along the locking channel 38, from the locking portion 42 toward the guide portion 40. In particular, rotating of the introducer hub 30 in the second direction directs the guide 31 out of the locking portion 42 of the locking channel 38 and through the guide portion 40 to release the introducer hub 30 from the sheath locking sleeve 28. Where the locking channel 38 includes a catch 44, rotation of the introducer hub 30 in the second direction causes the guide 31 to overcome the bias force of the catch 44 and advance from the locking portion 42 to the guide portion 40 of the locking channel 38. As a result, the guide 31 slides out of the locking channel 38 into the unlocked position. The introducer hub 30 is then disengaged from the sheath locking sleeve 28 and the introducer 6 can be withdrawn from the central lumen of the sheath 8.

A method of using a loader to insert a prosthetic device into a sheath hub will now be described. The method is described with reference to the components of FIGS. 24-26 for ease of description. However, it should be understood that the method could be used with variations to the structure of the loader assembly and sheath hub.

The method includes inserting a delivery system 10 including a catheter (such as balloon catheter 16) through a loader cap 304. The method can include inserting a proximal or a distal end of the catheter through a central seal 306 of the loader cap 304, as shown in FIG. 25A. The loader cap 304 is then positioned adjacent to a prosthetic device (such as heart valve 319), the prosthetic device located on a distal region of the catheter. For example, the loader cap 304 can be slid along the catheter of delivery system 10 until it is adjacent to the prosthetic device, as shown in FIG. 25B. Some versions of the method include mounting a prosthetic heart valve 319 onto the distal region of the catheter. The step of mounting or crimping the heart valve 319 can be performed before or after the step of positioning the loader cap 304 on to the delivery system 10.

The distal end of the balloon catheter 16 carrying the prosthetic device is inserted into a loader lumen via proximal end 321 of loader base 302. Proximal end 321 of the base is then fastened to the loader cap 304, thereby enclosing the prosthetic device within the loader lumen of loader assembly 300, as shown in FIG. 25B. For example, proximal end 321 of the base 302 can be fastened to the loader cap 304 by screwing the proximal end 321 of the base 302 to a threaded surface 316 on the loader cap 304.

Next, distally positioned tube 309 of the loader assembly 300 is inserted into a proximal locking sleeve (sheath locking sleeve 28) of the sheath hub 20. The tube 309 is pushed through an interior seal assembly of the sheath hub 20. A distal region 315 of loader base 302 is then pushed into the proximal sheath locking sleeve 28 of the sheath hub 20 until the proximal sleeve 28 abuts a step 317 on an outer surface of base 302, as shown in FIG. 26. The base 302 is thus rotationally and axially secured to sheath hub 20 via a press fit engagement between distal region 315 and the proximal sleeve 28 of the sheath hub 20.

Finally, the method further includes pushing distally the portion of delivery system 10 that extends proximally out of loader assembly 300. Pushing the delivery system 10 distally pushes the heart valve 319 through the tube 309 of the loader assembly 300, into the sheath 8, and eventually into the patient's vasculature.

In this example, a medical device is introduced into the proximal end of the central lumen of the expandable sheath 8. As provided in the example sheath 8 described above in reference to FIGS. 11-23, the sheath 8 includes a continuous inner layer (e.g., inner layers 102, 104, 106, 202) defining the central lumen extending through the sheath 8, an outer layer (e.g., outer layer 108, outer layer 204) provided over the inner layer, and a tubular strain relief layer 26 provided over the outer layer of the sheath 8. The strain relief layer 26 is provided at the proximal end of the sheath 8 and extends along a least a portion of the length of the sheath 8.

The medical device is advanced through a portion of the sheath 8 corresponding to the strain relief layer 26. The medical device is then advanced beyond the distal end of the strain relief layer 26 and into the lumen of the longitudinally body portion of the sheath 8 beyond the strain relief layer 26. As the medical device is advanced through the sheath 8 (beyond the strain relief layer 26), the sheath 8 locally expands from the unexpanded configuration (FIGS. 11-13A, 17-19) to the expanded configuration (FIGS. 14, 20) at a location proximate the medical device in response to the outwardly directed radially force of the medical device exerted against the inner layer/central lumen of the sheath 8.

As the medical device passes through the lumen of the sheath 8, the sheath 8 locally contracts at least partially back to the unexpanded configuration (FIGS. 11-13A, 17-19). When used to deliver a medical device to a treatment site within a patient, the medical device is then passed through the distal tip 9/distal opening of the sheath 8 and delivered to the treatment site. The position of the medical device can be moved or adjusted until the medical device is adequately positioned within the patient. With the medical device delivered to the treatment site, any delivery system/components coupled to the medical device are then removed from the medical device and withdrawn from the lumen of the sheath 8. The sheath 8 is removed from the patient and the opening in the blood vessel and skin closed.

In some examples, at least one of the inner layer and/or outer layer includes at least one folded portion, e.g., ridges 126 and valleys 128 of the fourth (outer) layer 108 of the sheath 8 illustrated in FIGS. 11-14, and folded portion 218 of the inner layer 202 of the sheath 8 illustrated in FIGS. 15-23. Locally expanding the lumen of the sheath 8 causes a length of the folded portion to at least partially unfold. Similarly, locally contracting the sheath 8 at least partially back to the unexpanded configuration causes a length of the folded portion to urge back towards a folded configuration.

In some examples, the outer layer is a discontinuous outer layer and includes an overlapping portion (e.g., overlapping portion 220) and an underlying portion (e.g., underlying portion 220). When the sheath 8 is in the unexpanded configuration, the overlapping portion overlaps the underlying portion with the folded portion of the inner layer disposed between the overlapping portion and the underlying portion (FIGS. 17, 19, 22, 23). As the sheath 8 locally expands to/toward the expanded configuration, a length of the overlapping portion moves circumferentially with respect to the underlying portion unfolding. As illustrated in FIG. 20, when the sheath 8 is fully expanded, the inner layer extends into the gap 232 formed between the longitudinal edges of the overlapping portion 220 and the underlying portion 222 of the outer layer 204.

In some examples, the sheath 8 includes an elastic outer layer 250 that extends at least partially over the outer layer and/or the strain relief layer 26. The elastic outer layer 250 locally expands and contracts as the medical device is advanced through the lumen of the sheath 8. In some examples, the elastic outer layer 250 urges the various layers of the sheath 8 toward an unexpanded configuration.

The medical device described above can include a prosthetic device mounted in a radially crimped state on a delivery apparatus, and the act of advancing the prosthetic device through the lumen of the sheath 8 comprises advancing the delivery apparatus and the prosthetic device through lumen of the sheath 8 and into the vasculature of the patient. In some examples, the prosthetic device comprises a prosthetic heart valve and the method further comprises implanting the prosthetic heart valve at a treatment site within the patient. As described above, the prosthetic heart valve is mounted on a balloon catheter of the delivery apparatus as the prosthetic heart valve is advanced through the sheath 8.

In view of the many possible aspects to which the principles of the disclosed disclosure can be applied, it should be recognized that the illustrated aspects are only preferred examples of the disclosure and should not be taken as limiting the scope of the disclosure. Rather, the scope of the disclosure is defined by the following claims. We, therefore, claim as our disclosure all that comes within the scope and spirit of these claims.

### Exemplary Aspects

Example 1: An assembly for delivering a prosthetic implant, the assembly comprising: a sheath hub comprising a proximal sleeve and an interior seal assembly; a sheath coupled to and extending distally from the sheath hub; and a loader comprising a base, a tube extending distally from the base and comprising a material that is different than a material of the base, and a loader lumen extending through the base and the tube; wherein the base of the loader comprises a proximal region, a central region, a distal region with an outer diameter narrower than an outer diameter of the central region, and a step defining a transition between the central region and the distal region; wherein the distal region of the base slides into the proximal sleeve of the sheath hub to engage in a press-fit configuration; and wherein when the assembly is in the press-fit configuration, the tube of the loader opens the interior seal assembly of the sheath hub.

Example 2: The assembly of any of the examples herein, particularly example 1, wherein the material of the tube is a lower durometer than the material of the base.

Example 3: The assembly of any of the examples herein, particularly examples 1-2, wherein the material of the tube is configured to withstand insertion through the interior seal assembly of the sheath hub.

Example 4: The assembly of any of the examples herein, particularly examples 1-3, wherein a portion of the tube is coaxially positioned within the base of the loader.

Example 5: The assembly of any of the examples herein, particularly examples 1-4, wherein the tube defines at least a portion of the loader lumen.

Example 6: The assembly of any of the examples herein, particularly examples 1-5, wherein the tube has a narrower outer diameter than an outer diameter of the base as measured adjacent a distal end of the base.

Example 7: The assembly of any of the examples herein, particularly examples 1-6, wherein when the assembly is in the press-fit configuration, the tube of the loader extends no further than a distal end of the sheath hub.

Example 8: The assembly of any of the examples herein, particularly examples 1-7, wherein the loader further comprises a removably attachable loader cap.

Example 9: The assembly of any of the examples herein, particularly examples 1-8, wherein the loader cap comprises a central seal.

Example 10: The assembly of any of the examples herein, particularly examples 1-9, wherein the proximal region of the base comprises threading, and the loader cap comprises a threaded surface to engage the threading of the base.

Example 11: The assembly of any of the examples herein, particularly examples 1-10, wherein the threading of the base is exterior threading, and the threaded surface of the loader cap is a threaded inner surface.

Example 12: The assembly of any of the examples herein, particularly examples 1-11, wherein the loader lumen is sized and configured to house a prosthetic heart valve.

Example 13: The assembly of any of the examples herein, particularly examples 1-12, wherein the assembly further comprises a prosthetic heart valve housed within the loader lumen.

Example 14: The assembly of any of the examples herein, particularly examples 1-13, wherein an inner diameter of the base widens approaching a proximal end of the base.

Example 15: The assembly of any of the examples herein, particularly examples 1-14, wherein the distal region of the base measures from 2 millimeters to 20 millimeters axially between the step and a distal end of the base.

Example 16: The assembly of any of the examples herein, particularly examples 1-15, wherein the outer diameter of the distal region proximate the step is from 0.3 millimeters to 6 millimeters smaller than the outer diameter of the central region proximate the step.

Example 17: The assembly of any of the examples herein, particularly examples 1-16, wherein the outer diameter of the distal region of the base is slightly larger than the inner diameter of the proximal sleeve of the sheath hub.

Example 18: The assembly of any of the examples herein, particularly examples 1-17, wherein the proximal sleeve of the sheath hub has a generally cylindrical inner surface and the distal region of the base has a generally cylindrical outer surface.

Example 19: The assembly of any of the examples herein, particularly examples 1-18, wherein the interior seal assembly reversibly closes a hub lumen that extends from a proximal end of the sheath hub to a distal end of the sheath hub.

Example 20: The assembly of any of the examples herein, particularly examples 1-19, wherein the interior seal assembly comprises a proximal seal, an intermediate seal, and a distal seal.

Example 21: A method for delivering a prosthetic device, the method comprising: positioning and stabilizing a sheath within a target location such that a sheath hub coupled to the sheath is adjacent to the target location; inserting a catheter through a loader cap; positioning the loader cap adjacent to the prosthetic device, the prosthetic device mounted on a distal region of the catheter; inserting the prosthetic device and the distal region of the catheter into a loader lumen extending through a loader; fastening a proximal end of a base of the loader to the loader cap to enclose the prosthetic device within the loader lumen; inserting a distally positioned tube of the loader into a proximal sleeve of the sheath hub; pushing the tube of the loader through an interior seal assembly of the sheath hub; pushing a distal region of the base into the proximal sleeve of the sheath hub until the proximal sleeve abuts a step on an outer surface of the base, thereby rotationally and axially securing the base to the sheath hub; pushing the catheter and the prosthetic device through the loader lumen; and pushing the catheter and the prosthetic device through the sheath.

Example 22: The method of any of the examples herein, particularly example 21, wherein the distal region of the base forms a press-fit engagement with the proximal sleeve of the sheath hub.

Example 23: The method of any of the examples herein, particularly examples 21-22, further comprising mounting the prosthetic device to the distal region of a catheter before inserting the prosthetic device and distal region of the catheter into the loader lumen.

Example 24: The method of any of the examples herein, particularly examples 21-23, wherein the target location is an access site to a vascular system of a patient.

Example 25: The method of any of the examples herein, particularly examples 21-24, wherein the prosthetic device is a heart valve.

Example 26: The method of any of the examples herein, particularly examples 21-25, wherein the catheter is inserted through a central seal of the loader cap.

Example 27: The method of any of the examples herein, particularly examples 21-26, wherein positioning the loader cap adjacent to a prosthetic device comprises sliding the loader cap along the catheter until it is adjacent the prosthetic device.

Example 28: The method of any of the examples herein, particularly examples 21-27, wherein fastening the proximal end of the base to the loader cap comprises screwing the proximal end of the base to a threaded surface on the loader cap.

What is claimed is:

1. An assembly for delivering a prosthetic implant, the assembly comprising:
a sheath hub comprising a proximal sleeve and an interior seal assembly;
a sheath coupled to and extending distally from the sheath hub; and
a loader comprising a base, a tube extending distally from the base and comprising a material that is different than a material of the base, and a loader lumen extending through the base and the tube;
wherein the base of the loader comprises a proximal region, a central region, a distal region with an outer diameter narrower than an outer diameter of the central region, and a step defining a transition between the central region and the distal region;
wherein the distal region of the base slides into the proximal sleeve of the sheath hub to engage in a press-fit configuration; and
wherein when the assembly is in the press-fit configuration, the tube of the loader opens the interior seal assembly of the sheath hub.

2. The assembly of claim 1, wherein the material of the tube is a lower durometer than the material of the base and configured to withstand insertion through the interior seal assembly of the sheath hub.

3. The assembly of claim 1, wherein a portion of the tube is coaxially positioned within the base of the loader.

4. The assembly of claim 1, wherein the tube defines at least a portion of the loader lumen.

5. The assembly of claim 1, wherein the tube has a narrower outer diameter than an outer diameter of the base as measured adjacent a distal end of the base.

6. The assembly of claim 1, wherein when the assembly is in the press-fit configuration, the tube of the loader extends no further than a distal end of the sheath hub.

7. The assembly of claim 1, wherein the loader further comprises a removably attachable loader cap, the loader cap including a central seal.

8. The assembly of claim 1, wherein the proximal region of the base comprises threading, and the loader cap comprises a threaded surface to engage the threading of the base, wherein the threading of the base is exterior threading, and the threaded surface of the loader cap is a threaded inner surface.

9. The assembly of claim 1, wherein the loader lumen is sized and configured to house a prosthetic heart valve,
wherein the assembly further comprises a prosthetic heart valve housed within the loader lumen.

10. The assembly of claim 1, wherein an inner diameter of the base widens approaching a proximal end of the base.

11. The assembly of claim 1, wherein the outer diameter of the distal region of the base is slightly larger than an inner diameter of the proximal sleeve of the sheath hub.

12. The assembly of claim 1, wherein the proximal sleeve of the sheath hub has a generally cylindrical inner surface and the distal region of the base has a generally cylindrical outer surface.

13. The assembly of claim 1, wherein the interior seal assembly reversibly closes a hub lumen that extends from a proximal end of the sheath hub to a distal end of the sheath hub,
wherein the interior seal assembly comprises a proximal seal, an intermediate seal, and a distal seal.

14. A method for delivering a prosthetic device, the method comprising:
positioning and stabilizing a sheath within a target location such that a sheath hub coupled to the sheath is adjacent to the target location;
inserting a catheter through a loader cap;
positioning the loader cap adjacent to the prosthetic device, the prosthetic device mounted on a distal region of the catheter;
inserting the prosthetic device and the distal region of the catheter into a loader lumen extending through a loader;
fastening a proximal end of a base of the loader to the loader cap to enclose the prosthetic device within the loader lumen;
inserting a distally positioned tube of the loader into a proximal sleeve of the sheath hub;
pushing the tube of the loader through an interior seal assembly of the sheath hub;
pushing a distal region of the base into the proximal sleeve of the sheath hub until the proximal sleeve abuts a step on an outer surface of the base, thereby rotationally and axially securing the base to the sheath hub;
pushing the catheter and the prosthetic device through the loader lumen; and
pushing the catheter and the prosthetic device through the sheath.

15. The method of claim 14, wherein the distal region of the base forms a press-fit engagement with the proximal sleeve of the sheath hub.

16. The method of claim 14, further comprising mounting the prosthetic device to the distal region of a catheter before inserting the prosthetic device and distal region of the catheter into the loader lumen.

17. The method of claim 14, wherein the target location is an access site to a vascular system of a patient and the prosthetic device is a heart valve.

18. The method of claim 14, wherein the catheter is inserted through a central seal of the loader cap.

19. The method of claim 14, wherein positioning the loader cap adjacent to a prosthetic device comprises sliding the loader cap along the catheter until it is adjacent the prosthetic device.

20. The method of claim 14, wherein fastening the proximal end of the base to the loader cap comprises screwing the proximal end of the base to a threaded surface on the loader cap.
